(19) United States
(12) Patent Application Publication
(10) Pub. No.: US 2023/0109713 A1
(43) Pub. Date: Apr. 13, 2023

(54) METHODS AND SYSTEMS FOR LEVITATION-BASED MAGNETIC SEPARATION

(71) Applicant: LevitasBio, Inc., Menlo Park, CA (US)

(72) Inventors: Kathy Lazaruk, Redwood City, CA (US); Christopher Stevens, Fremont, CA (US); Kevin Travers, Menlo Park, CA (US)

(73) Assignee: LevitasBio, Inc., Menlo Park, CA (US)

(21) Appl. No.: 18/045,473

(22) Filed: Oct. 11, 2022

Related U.S. Application Data
(60) Provisional application No. 63/362,627, filed on Apr. 7, 2022.
(60) Provisional application No. 63/254,946, filed on Oct. 12, 2021.

(51) Int. Cl. B03C 1/01; B03C 1/28; B82Y 30/00

(52) U.S. Cl. CPC B03C 1/01; B03C 1/28; B82Y 30/00; B03C 2201/18; B03C 2201/26

(57) ABSTRACT

Described are various embodiments of methods, devices, systems and kits for magnetic levitation-based separation of mixtures or populations of particles that include various types of particles. Some embodiments of such methods, devices, systems and kits are useful for magnetic levitation-based separation of mixtures or populations of cells that include various cell types. Some other embodiments of the described methods, devices, systems and kits are useful for magnetic levitation-based separation of mixtures or population of cellular or mixtures or population of biological molecules.

### CROSS-REFERENCES TO RELATED APPLICATIONS

This application claims the benefit of U.S. Provisional Application No. 63/362,627, filed Apr. 7, 2022, and U.S. Provisional Application 62/254,946 filed Oct. 12, 2021. The entire contents and disclosures of these applications are incorporated herein by reference.

### BACKGROUND OF THE INVENTION

Magnetic levitation recently emerged as a useful method for separating particles, including cells and biological molecules. During magnetic levitation a particle suspended in a paramagnetic fluid medium is exposed to a magnetic field gradient, which generates a nonuniform pressure equivalent to the magnetic energy density in the paramagnetic fluid medium. In a magnetic field gradient, the particles (or “objects”) subjected to magnetic levitation appear to be repelled from the regions of high magnetic field. In actuality, the object is displaced by an equal volume of the paramagnetic fluid medium. The attractive interaction between the paramagnetic fluid medium and the regions of high magnetic field results in magnetic levitation of the object. Conditions and systems have been established for levitating live cells, and it has been demonstrated that both eukaryotic and prokaryotic cells have unique magnetic levitation profiles. See Durmus et al., 2015, “Magnetic levitation of single cells,” Proc Natl Acad Sci USA 112(28):E3661-8 (“Durmus”). Systems for collecting cells at different levitation heights and collecting them for downstream analysis and other applications have been developed.

### BRIEF SUMMARY OF THE INVENTION

The terms “invention,” “the invention,” “this invention” and “the present invention,” as used in this document, are intended to refer broadly to all of the subject matter of this patent application and the claims below. Statements containing these terms should be understood not to limit the subject matter described in the present disclosure or to limit the meaning or scope of the patent claims below. Covered embodiments of the invention are defined by the claims, not this summary. This summary is a high-level overview of various aspects of the invention and introduces some of the concepts that are described and illustrated in the present document and the accompanying figures. This summary is not intended to identify key or essential features of the claimed subject matter, nor is it intended to be used in isolation to determine the scope of the claimed subject matter. The subject matter should be understood by reference to appropriate portions of the entire specification, any or all figures and each claim. Some of the exemplary embodiments of the present invention are discussed below.

Included among the embodiments of the present invention and described in the present disclosure are methods of cell separation. Some embodiments of the methods comprise the steps of: binding a first levitation-height altering agent to a cell of a first type in a population of cells comprising multiple cell types, wherein the first levitation-height altering agent comprises a first paramagnetic or superparamagnetic microparticle and a first linking agent that preferentially binds to cells of the first type, thereby forming a first complex, said first complex comprising the first levitation-height altering agent bound to an individual cell of the first type; forming a suspension in a paramagnetic fluid medium, the suspension comprising a plurality of the first complexes and a plurality of the cells of the multiple cell types; introducing the suspension into a processing channel of a flowcell cartridge; and, exposing the processing channel to a magnetic field for a first period of time sufficient for at least some of the first complexes to separate in the processing channel from the cells of the multiple cell types not bound by the first levitation-height altering agent, thereby forming a first portion of the suspension, wherein the first portion is enriched with the first complex relative to the suspension, and a second portion of the suspension, the second portion depleted of the first complex relative to the suspension. Some embodiments of the methods comprise the steps of: combining a magnetic agent and population of cells comprising multiple cell types, wherein the magnetic agent comprises a magnetic microparticle and a linking agent that preferentially binds to cells of a target type of the multiple cell types, thereby forming a magnetic complex, said magnetic complex comprising the magnetic agent bound to an individual cell of the target type; forming a suspension in a paramagnetic fluid medium, the suspension comprising a plurality of the magnetic complexes and a plurality of the cells of the multiple cell types; introducing the suspension into a processing channel of a flowcell cartridge; and, exposing the processing channel to a magnetic field for period of time sufficient for at least some of the plurality of the magnetic complexes to migrate to and be immobilized against one or more sides of the processing channel, thereby forming a suspension depleted of the magnetic complex.

Also included among the embodiments of the present invention and described in the present disclosure are kits for magnetic levitation. For example, a magnetic levitation kit may comprise a paramagnetic fluid medium and one or more levitation-height altering agents, or separate components of the one or more of the levitation-height altering agents, capable of forming complexes with individual cells, wherein each levitation-height altering agent comprises a paramagnetic or superparamagnetic microparticle, and a linking agent that preferentially binds to a target cell type. In another example, a magnetic levitation kit may comprise a paramagnetic fluid medium and one or more magnetic agents, or separate components of the one or more of the magnetic agents, capable of forming complexes with individual cells, wherein each magnetic agent comprises a magnetic microparticle, and a linking agent that preferentially binds to a target cell type. Also included among the embodiments of the present invention are systems for cell separation. An exemplary system may comprise a magnetic microparticle capable, alone or in combination with other reagents, of preferentially binding to cells of a first type in a population of cells comprising multiple cell types and forming a first complex of the microparticle and a cell of the first type; a flowcell cartridge comprising a first outlet channel and a processing channel; a station comprising a holding block for the flowcell cartridge and one or more magnets positioned to expose the processing channel of the flowcell cartridge located in the holding block to a magnetic field, wherein exposing to the magnetic field the processing channel of the flowcell cartridge containing a suspension of the cells of the multiple cell types in a paramagnetic fluid medium allows the first complex to separate in the processing channel from the cells of the multiple cell types not bound by the first non-magnetic microparticle and from the other types of cells of the multiple cell types, and, wherein the first complex levitates lower in the processing channel of the flowcell cartridge is lower than the multiple cell types not complexed in the magnetic particle.

### BRIEF DESCRIPTION OF THE DRAWINGS

FIG. 1 is a schematic representation of an exemplary magnetic levitation system.

FIG. 2 is a schematic representation of two views of an exemplary flowcell cartridge of a magnetic levitation system.

FIG. 3 is a schematic representation of a method according to an exemplary embodiment of the present invention.

FIG. 4 is a schematic representation of a method according to an exemplary embodiment of the present invention.

FIG. 5 are photographic images illustrating separation of cells complexed to magnetic microbeads according to some embodiments described the present disclosure.

FIG. 6 are dot plots illustrating the effects of antibody surface coverage on cell separation according to an exemplary embodiment of the present invention.

FIG. 7 is a schematic illustration of the model of the microparticles complexed to a cell surface.

FIG. 8 is a table illustrating density calculation for microparticle-cell complexes, with the microparticles having a density of 1.063 g/cm³, and the cell having a diameter of 11.5 µm.

### DETAILED DESCRIPTION OF THE INVENTION

Cells have inherent properties that dictate their behavior when subjected to magnetic levitation. Durmus et al. showed that the height at which a cell levitates in a paramagnetic fluid medium (“levitation profile”) corresponds to cellular density, and that different cell types can be distinguished based on their characteristic magnetic levitation profiles. See also co-pending, commonly owned U.S. Pat. Application No. 17/449,438, filed Sep. 29, 2021, incorporated by reference herein, which describes methods and systems for separation of cells by magnetic levitation, including the methods for separation of dead cells from living cells. Described in the present disclosure are improved processes (methods), devices, systems and kits for separation of particles, including cells, by magnetic levitation.

The inventors discovered methods that improve separation of particles, such as cells, during a magnetic levitation process. The inventors found that, by binding cells to paramagnetic or superparamagnetic microparticles, they were able to alter the levitation height of the cells, when the cells were suspended in a paramagnetic fluid medium in a flowcell cartridge of a magnetic levitation system and exposed to a magnetic field. Levitation height may be defined by vertical position of the cells in a flowcell cartridge of a magnetic levitation system. In other words, the inventors were able to control the levitation height of the cells during magnetic levitation process by complexing the cells with paramagnetic or superparamagnetic microparticles. In one example, cells with cell-specific surface markers were bound to superparamagnetic microparticles coupled to anti-surface marker antibodies. The resulting complexes of the cells with superparamagnetic microparticles (each complex including one cell with cell-specific surface markers bound to one or more superparamagnetic microparticles) were suspended in a paramagnetic medium, and magnetically levitated in a processing channel of a flowcell cartridge of a magnetic levitation system.

In the above example, the levitation height of the complexes of cells with superparamagnetic microparticles was affected by the number of the superparamagnetic particles in each complex. Depending on the number of superparamagnetic microparticles linked to each cell, the complexes either “dropped out” (that is, immobilized at the bottom of the processing channel, resulting in “depletion” of the paramagnetic medium of the complexes) or levitated in the processing channel, presumably because the magnetic force in the processing channel was not strong enough to pull the complexes to the bottom of the channel. In a series of experiments, the number of superparamagnetic particles per cell in the complexes was varied by changing the ratio of particles to cells during complex formation (“PTC ratio”) from about 1 to about 100,000. The inventors found that increasing the PTC ratio lowered the levitation height of the complexes. When the PTC ratio was in the range of about 10,000-50,000, the complexes dropped to the bottom of the processing channel of the flowcell cartridge, effectively achieving the depletion of the paramagnetic medium of the complexes. When the PTC ratio was in the range of about 1-1,000, the complexes levitated lower in the processing channel than the same cells not complexed to the superparamagnetic microparticles, but did not drop to the bottom of the processing channel. In view of the above findings, the inventors realized that, in some cases, it may be beneficial to use smaller paramagnetic or superparamagnetic microparticles in order to achieve higher concentration during complex formation, which may result in increased PTC ratios. The inventors also found that various other parameters, in addition to the PTC ratio and the applied magnetic field strength, affected the levitation height. Some of these parameters are the properties of materials included in paramagnetic or superparamagnetic microparticle (which may affect magnetic susceptibility of the microparticle), microparticle size, and microparticle density.

Thus, by coupling paramagnetic or superparamagnetic microparticles to antibodies specific to surface markers characteristic of a cell type of interest found in a population of cells containing multiple cell types and choosing one or more parameters affecting the levitation height, the inventors were able to selectively alter levitation height of the cells belonging to the cell type of interest. As discussed in detail in the present disclosure, paramagnetic or superparamagnetic microparticles may be used to segregate, by magnetic levitation, specified cells from a mixed population of cells suspended in a paramagnetic medium, resulting in fractions enriched for or depleted of the cell type of interest. These fractions can be then withdrawn from the flowcell of the magnetic levitation system, accomplishing separation of a cell type of interest. As also described in the present disclosure, paramagnetic or superparamagnetic microparticles may be used to separate, by magnetic levitation various components of interest from various types of heterogeneous mixtures, such as separation of cell organelles, nucleic acids, or other molecules. Components of interest (e.g., cells, cell organelles, nucleic acids) are sometimes referred to as “analytes.”

As also discussed in detail in the present disclosure, magnetic microparticles (which include nanoparticles, and may be ferromagnetic, ferrimagnetic, paramagnetic, or superparamagnetic) may be used to selectively deplete a mixed population of analytes (such as cells or other particles) suspended in a paramagnetic medium of a specific analyte (such as a cell type or other type particle) by forming the complexes of magnetic particles and the specific analyte and allowing them to migrate and adhere to one or more sides of the processing channel of the magnetic levitation cartridge under the influence of the magnetic force during magnetic levitation. The paramagnetic fluid medium depleted of the analyte of interest can then be withdrawn from the processing channel.

Thus, the present disclosure describes various embodiments of methods, devices, systems and kits for magnetic levitation-based separation of mixtures or populations of particles that include various types of particles. Processes, devices, methods, and kits conceived by the inventors are useful for a variety of applications. Some embodiments of methods, devices, systems, and kits described in the present disclosure are useful for magnetic levitation-based separation of cells or subpopulations of cells from heterogeneous mixtures or populations of cells that include various cell types. Some other embodiments of methods, devices, systems, and kits described in the present disclosure are useful for magnetic levitation-based separation of mixtures or population of cellular organelles or other cellular components (including endocellular and exocellular components, for example, but not limited to, endosomes or exosomes). Yet some other embodiments of methods, devices, systems, and kits described in the present disclosure are useful for magnetic levitation-based separation of mixtures or population of biological molecules or complexes of molecules, such as separation of nucleic acids, for example, separation of nucleic acid libraries during next generation sequencing (NGS), or separation of lipoproteins. Some embodiments of methods, devices, systems, and kits described in the present disclosure are useful for magnetic levitation-based separation of mixtures or population separation of cells that have taken up by endocytosis magnetic particles.

Methods, devices, systems, and kits described in the present disclosure possess various advantages over previously known magnetic levitation-based separation methods. Some of these advantages are improved separation precision and speed, improved reproducibility, and the ability to separate complex multi-component mixtures. Some other advantages are the ability to levitate molecules that would otherwise be difficult to levitate to a specific position during magnetic levitation, one example being RNA (such as RNA released from lysed cells), and the ability to separate, by magnetic levitation, cell types that have very similar densities (and therefore, on their own, cannot be separated using magnetic levitation).

### Terms and Concepts

A number of terms and concepts are discussed below. They are intended to facilitate the understanding of various embodiments of the invention in conjunction with the rest of the present document and the accompanying figures. These terms and concepts may be further clarified and understood based on the accepted conventions in the fields of the present invention, as well as the description provided throughout the present document and/or the accompanying figures. Some other terms can be explicitly or implicitly defined in other sections of this document and in the accompanying figures and may be used and understood based on the accepted conventions in the fields of the present invention, the description provided throughout the present document and/or the accompanying figures. The terms not explicitly defined can also be defined and understood based on the accepted conventions in the fields of the present invention and interpreted in the context of the present document and/or the accompanying figures.

Unless otherwise dictated by context, singular terms shall include pluralities, and plural terms shall include the singular. Generally, nomenclatures used in connection with, and techniques of, cell and tissue culture, molecular biology, immunology, microbiology, genetics and protein and nucleic acid chemistry are those well-known and commonly used. Known methods and techniques are generally performed according to conventional methods well-known and as described in various general and more specific references, unless otherwise indicated. The nomenclatures used in connection with the laboratory procedures and techniques described in the present disclosure are those well-known and commonly used.

### Miscellaneous

As used in the present disclosure, the terms “a”, “an”, and “the” can refer to one or more unless specifically noted otherwise.

The use of the term “or” is used to mean “and/or,” unless explicitly indicated to refer to alternatives only, or the alternatives are mutually exclusive, although the disclosure supports a definition that refers to only alternatives and “and/or.” As used in the present disclosure “another” can mean at least a second or more.

As used in the present disclosure, and unless otherwise indicated, the terms “include,” “including,” and, in some instances, similar terms (such as “have” or “having”) mean “comprising.”

When a numerical range is provided in the present disclosure, the numerical range includes the range endpoints unless otherwise indicated. Unless otherwise indicated, numerical ranges in the present disclosure include all values and subranges, as if explicitly written out.

The terms “about” and “approximately,” as used in the present disclosure, shall generally mean an acceptable degree of error for the quantity measured, given the nature or precision of the measurements. Exemplary degrees of error are within 20%, 10%, 9%, 8%, 7%, 6%, 5%, 4%, 3%, 2%, or 1% of a given value or range of values. For example, any reference to “about X” or “approximately X” specifically indicates at least the values X, 0.9X, 0.91X, 0.92X, 0.93X, 0.94X, 0.95X, 0.96X, 0.97X, 0.98X, 0.99X, 1.01X, 1.02X, 1.03X, 1.04X, 1.05X, 1.06X, 1.07X, 1.08X, 1.09X, and 1.10X. In another example, the terms “about” or “approximately” in relation to a reference numerical value can include a range of values plus or minus 10%, 9%, 8%, 7%, 6%, 5%, 4%, 3%, 2%, or 1% from that value. Thus, expressions “about X” or “approximately X” are intended to describe a claim limitation of, for example, “0.98X.” Numerical quantities given in the present disclosure are approximate unless stated otherwise, meaning that the term “about” or “approximately” can be inferred when not expressly stated. When the terms “about” or “approximately” are applied to the beginning of a numerical range, they apply to both ends of the range. Where a series of values is prefaced with the terms “about” or “approximately,” these terms are intended to modify each value included in the series.

The terms “plurality” or “population,” when used in connection with particles, such as, but not limited to, cells (for example, as in “a plurality of cells” or “a population of cells”), refer to groups of particles (that is, more than one particle) including various numbers of particles. For example, a plurality or a population of particles, such as cells, may include 2 or more, 10 or more, 100 or more, 500 or more, 10³ or more, 10⁴ or more, 10⁵ or more, 10⁶ or more, or 10⁷, or more particles.

The terms “peptide,” “polypeptide” or “protein” are used to refer polymer of amino acids linked by native amide bonds and/or non-native amide bonds. Peptides, polypeptides or proteins may include moieties other than amino acids (for example, lipids or sugars). Peptides, polypeptides or proteins may be produced synthetically or by recombinant technology.

The terms “oligonucleotide,” “polynucleotide” or “nucleic acid” encompass DNA or RNA molecules, including the molecules produced synthetically or by recombinant technology. Oligonucleotides, polynucleotides or nucleic acids may be single-stranded or double-stranded.

The term “small molecule” includes molecules (either organic, organometallic, or inorganic), organic molecules, and inorganic molecules, respectively, which have a molecular weight of more than about 50 Da and less than about 2500 Da. Small organic (for example) molecules may be less than about 2000 Da, between about 100 Da to about 1000 Da, or between about 100 Da to about 600 Da, or between about 200 Da to about 500 Da.

The term “paramagnetic” and the related terms and expressions refer in the present disclosure to materials and particles displaying paramagnetic properties, or paramagnetism. Paramagnetism is a form of magnetism characteristic of materials weakly attracted by an externally applied magnetic field. Paramagnetic materials do not retain any magnetization in the absence of an externally applied magnetic field. See, for example, Britannica, The Editors of Encyclopaedia. “Paramagnetism”. Encyclopedia Britannica, 20 Dec. 2006. In other words, paramagnetic materials have small susceptibility to magnetic field (“magnetic susceptibility”), but do not retain magnetic properties once magnetic field is removed.

The term “superparamagnetic” and the related terms and expressions refer in the present disclosure to microparticles displaying superparamagnetic properties, or superparamagnetism. Superparamagnetism is a phenomenon observed in small ferromagnetic or ferrimagnetic particles. If the size of these particles is small enough (in the nanoparticle size range), their magnetization can randomly flip direction under the influence of temperature. The time between two flips is known as the Néel relaxation time. If the time used to measure the magnetization of the microparticles is much longer than the Néel relaxation time, and no external field is present, their average magnetization seems to be zero, and they are said to be in superparamagnetic state. See Pedro M. Enriquez-Navas and Maria L.Garcia-Martin “Application of Inorganic Nanoparticles for Diagnosis Based on MRI” Frontiers of Nanoscience 4:233-245 (2012) doi.org/10.1016/B978-0-12-415769-9.00009-1. Superparamagnetic materials exhibit magnetic susceptibility in externally applied magnetic field, but do not without such field.

### Separation, Isolation, and Concentration

As used in the present disclosure, the term “concentration” means an amount of a first component contained within a second component, and may be based on the number of particles per unit volume, a molar amount per unit volume, weight per unit volume, or based on the volume of the first component per volume of the combined components.

As used in the present disclosure, the terms “isolate,” “separate,” “segregate,” “purify,” and their respective related terms and expressions may be used interchangeably. These terms may be used to refer to a procedure that enriches the amount of one or more components of interest relative to one or more other components present in a sample. In reference to a particle or a component (which may be a cell), such terms may mean one or more of: separating such component from other components, increasing the concentration of a component within a solution, or separating a component from other components in a solution. For example, a particle within a solution may be deemed “isolated,” if it is segregated from other particles within the solution and/or positioned within a defined portion of the solution. In another example, a particle or component within a solution is deemed “isolated,” if, after processing the solution, the concentration of such particle or component is increased by a ratio of at least about 100:1, 90:1, 80:1, 70:1, 60:1, 50:1, 40:1, 30:1, 20:1, 10:1, 5:1, 3:1 2:1, 1.5:1 or 1.1:1. Particles of interest within a solution containing multiple types of particles may be deemed “separated” if, after processing the solution, the ratio of the concentration of the particles of interest to the concentration of other types of particles is increased, or if the ratio of the concentration of the particles of interest to the concentration of other types particles is increased by at least about 10%, 50%, 100%, 200%, 300%, 400%, 500%, 600%, 700%, 800%, 900%, or 1000%,, or if the concentration of other components of the solution (including, but not limited to, the types of particles other than the particles of interest) is decreased to at least about 80%, 70%, 60%, 50%, 40%, 30% 20%, 15%, 10%, 8%, 6%, 5%, 4%, 3%, 2%, 1%, or 0.5%.

### Levitation-Height Altering Agent; Magnetic Agent

A levitation-height altering agent (that is, an agent capable of altering levitation height of a particle during magnetic levitation) comprises (i) a magnetic (paramagnetic or superparamagnetic) microparticle and (ii) a linking agent that preferentially binds to particles, (such as, but not limited to, cells), of the specified (e.g., first) type. The levitation-height altering agent, by virtue of the linking agent, similarly preferentially binds to particles (such as, but not limited to cells) of the specified (e.g., first) type. Levitation-height altering agents are sometimes referred to as “tags.” The process of linking levitation-height altering agents or magnetic particles to cells (or other particles) is sometimes referred to as “tagging.”

Intrinsic characteristic capacity of a levitation-height altering agent to alter, under a specified set of conditions levitation height of particles with which levitation-height altering agent forms complexes, can be referred to as the levitation-height altering agent’s “levitation height-altering property,” “levitation height-altering properties,” or by other related terms and expressions. Levitation height-altering properties of a levitation-height altering agent can be influenced, for example, by the materials included in the magnetic microparticles of the levitation-height altering agent, microparticle size, and microparticle density

A magnetic agent (that is, an agent capable of adhering a particle to one or more sides of a processing channel of a flowcell cartridge during magnetic levitation) comprises (i) a magnetic (ferromagnetic, ferromagnetic, paramagnetic, or superparamagnetic) microparticle and (ii) a linking agent that preferentially binds to particles (such as, but not limited to, cells) of the specified (e.g., first) type. The magnetic agent, by virtue of the linking agent, similarly preferentially binds to particles (such as, but not limited to cells) of the specified (e.g., first) type. Magnetic agents and/or levitation-height altering agent can be referred to as “tags” or “magnetic tags.” The process of linking levitation-height altering agent or agents to cells (or other particles) is sometimes referred to as “tagging” or “magnetic tagging.” It will be understood that, in some situations, the terms “magnetic agent” and “levitation-height altering agent” may be used to refer to an agent comprising the same type of magnetic microparticles, as various parameters discussed elsewhere in the present disclosure affect how a cell (or other particle) tagged with a particular agent will behave during magnetic levitation-based separation process (for example, will the complex levitate lower than the untagged cells or particles in the mixture, or will it “drop out” of the mixture to the bottom of the processing channel of the flowcell cartridge).

### Magnetic Microparticle

The terms “microparticle,” “microsphere,” and “microbead” (which encompass, respectively, a “nanosphere,” “nanosphere,” “nanoparticle,” and “nanobead”) refer, in the context of the present disclosure, to magnetic (including ferrimagnetic, ferromagnetic paramagnetic and superparamagnetic) particles having one or more dimensions (such as length, width, diameter, or circumference) of about 500 µm or less (as discussed below). A microparticle may have a generally spherical shape or a non-spherical shape.

Microparticles (including ferrimagnetic, ferromagnetic paramagnetic and superparamagnetic microparticles, encompassing nanoparticles) used in the embodiments of the present invention can have a range of sizes. For example, a microparticle (which can be a nanoparticle), such as a superparamagnetic microparticle (which can be a superparamagnetic nanoparticle) or a paramagnetic microparticle (which can be a paramagnetic nanoparticle), may have a cross-sectional dimension (e.g., diameter, length, width) about 500 µm or less, about 100 µm or less, about 50 µm or less, about 20 µm or less, about 10 µm or less, about 5 µm or less, about 1 µm (1000 nm) or less, about 0.5 µm (500 nm) or less, about 0.25 µm (250 nm) or less, about 0.1 µm (100 nm) or less about 0.05 µm (50 nm) or less, or about 0.025 µm (25 nm) or less, such as a cross-sectional dimension in the range of about 500 µm to about 0.0005 µm (0.5 nm), 500 µm to about 0.001 µm (1 nm), about 500 µm to about 0.01 µm (10 nm), about 500 µm to about 0.025 µm (25 nm), about 500 µm to about 0.05 µm (50 nm), about 500 µm to about 0.1 µm (100 nm), about 500 µm to about 0.25 µm (250 nm), about 500 µm to about 0.5 µm (500 nm), about 500 µm to about 1 µm (1000 nm), about 500 µm to about 10 µm, about 500 µm to about 20 µm, about 500 µm to about 100 µm, about 100 µm to about 0.0005 µm (0.5 nm), about 100 µm to about 0.001 µm (1 nm), about 100 µm to about 0.01 µm (10 nm), about 100 µm to about 0.025 µm (25 nm), about 100 µm to about 0.05 µm (50 nm), about 100 µm to about 0.1 µm (100 nm), about 100 µm to about 0.25 µm (250 nm), about 100 µm to about 0.5 µm (500 nm), about 100 µm to about 1 µm (1000 nm), about 100 µm to about 10 µm, about 100 µm to about 20 µm, about 20 µm to about 0.0005 µm (0.5 nm), about 20 µm to about 0.001 µm (1 nm), about 20 µm to about 0.01 µm (10 nm), about 20 µm to about 0.025 µm (25 nm), about 20 µm to about 0.05 µm (50 nm), about 20 µm to about 0.1 µm (100 nm), about 20 µm to about 0.25 µm (250 nm), about 20 µm to about 0.5 µm (500 nm), about 20 µm to about 1 µm (200 nm), or about 20 µm to about 10 µm, about 10 µm to about 0.0005 µm (0.5 nm), about 10 µm to about 0.001 µm (1 nm), about 10 µm to about 0.01 µm (10 nm), about 10 µm to about 0.025 µm (25 nm), about 10 µm to about 0.05 µm (50 nm), about 10 µm to about 0.1 µm (100 nm), about 10 µm to about 0.25 µm (250 nm), about 10 µm to about 0.5 µm (500 nm), about 10 µm to about 1 µm (200 nm), about 5 µm to about 0.0005 µm (0.5 nm), about 5 µm to about 0.001 µm (1 nm), about 5 µm to about 0.01 µm (10 nm), about 5 µm to about 0.025 µm (25 nm), about 5 µm to about 0.05 µm (50 nm), about 5 µm to about 0.1 µm (100 nm), about 5 µm to about 0.25 µm (250 nm), about 5 µm to about 0.5 µm (500 nm), about 5 µm to about 1 µm (200 nm), about 1 µm to about 0.0005 µm (0.5 nm), about 1 µm to about 0.001 µm (1 nm), about 1 µm to about 0.01 µm (10 nm), about 1 µm to about 0.025 µm (25 nm), about 1 µm to about 0.05 µm (50 nm), about 1 µm to about 0.1 µm (100 nm), about 1 µm to about 0.25 µm (250 nm), about 1 µm to about 0.5 µm (500 nm), about 0.5 µm (500 nm) to about 0.0005 µm (0.5 nm), about 0.5 µm (500 nm) to about 0.001 µm (1 nm), about 0.5 µm (500 nm) to about 0.01 µm (10 nm), about 0.5 µm (500 nm) to about 0.025 µm (25 nm), about 0.5 µm (500 nm) to about 0.05 µm (50 nm), about 0.5 µm (500 nm) to about 0.1 µm (100 nm), about 0.5 µm (500 nm) to about 0.25 µm (250 nm), about 0.25 µm (250 nm) to about 0.0005 µm (0.5 nm), about 0.25 µm (250 nm) to about 0.001 µm (1 nm), about 0.25 µm (250 nm) to about 0.01 µm (10 nm), about 0.25 µm (250 nm) to about 0.025 µm (25 nm), about 0.25 µm (250 nm) to about 0.05 µm (50 nm), about 0.25 µm (250 nm) to about 0.1 µm (100 nm), about 0.1 µm (100 nm) to about 0.0005 µm (0.5 nm), about 0.1 µm (100 nm) to about 0.001 µm (1 nm), about 0.1 µm (100 nm) to about 0.01 µm (10 nm), about 0.1 µm (100 nm) to about 0.025 µm (25 nm), about 0.1 µm (100 nm) to about 0.05 µm (50 nm), about 0.05 µm (50 nm) to about 0.0005 µm (0.5 nm), about 0.05 µm (50 nm) to about 0.001 µm (1 nm), about 0.05 µm (50 nm) to about 0.01 µm (10 nm), or about 0.05 µm (50 nm) to about 0.025 µm (25 nm).

Magnetic microparticles (including ferrimagnetic, ferromagnetic paramagnetic and superparamagnetic microparticles, encompassing nanoparticles) used in the embodiments of the present invention can be composed of, or can comprise, any number of ferromagnetic, ferrimagnetic or paramagnetic materials or their combinations, including, but not limited to: a metal, such as, but not limited to, iron, magnesium or molybdenum, a metal salt, or a metal oxide (for example, iron oxide), suitable ceramics, and/or suitable composite materials, such as monodisperse nanoporous silica containing iron oxide particles within the porous silica network. One example of a magnetic microparticle used in the embodiments of the present invention is a microparticle that has a magnetic core (such as, but not limited to, Fe₂O₃ core) and a polymer coating (for example, a coating may be made of or comprising one or more of polystyrene, dextran, polyethylene glycol (PEG), polymethyl methacrylate (PMMA), or polyethylene. Some other examples of magnetic microparticle used in the embodiments of the present invention are dextran-coated magnetic nanoparticles, gold-coated magnetic nanoparticles or silica-coated magnetic nanoparticles, such as those described in U.S. Pat. No. 7,169,618.

It will be recognized that magnetic microparticles used in a single separation process according to the methods described in the present disclosure may include multiple (two or more) types of microparticles with different levitation height-altering and/or magnetic properties. In some exemplary embodiments, such different types of microparticles may be coupled to different linking agents. For example, one (first) type of magnetic microparticle used in a separation process may be coupled to a linking agent for tagging a first cell type (e.g., CD8+ T cells) and a different (second) type of magnetic microparticle may be coupled to liking agent for tagging a second cell type (e.g., CD4+ T cells). In some embodiments according to the present disclosure, levitation-height altering agents that comprise the same linking agent (or comprise linking agents with the same specificity) will be associated with microparticles with the same levitation-altering or magnetic properties.

### Factors Affecting Microparticle Selection

Binding of a levitation-height altering agent or a magnetic agent to cells or other objects and behaviors of the resulting complexes during magnetic levitation depends on several interconnected variables including, but not limited to, the density of the microparticles included in the density-modifying agent, the ratio of the microparticles to cells during complex formation (PTC ratio is discussed elsewhere in the present disclosure), microparticle size, the size of cells// or other objects being tagged, the ratio of the above sizes, the density of the linking agent on the microparticle surface, and microparticle material. The above and other factors affect microparticle selection for a particular separation process and may be estimated using theoretical calculations, some of which are discussed below.

In one example, the density of a complex of a cell is estimated as the sum of the mass of the cell (m_{cell}) and all bound microparticles (n*m_{MS}) divided by the sum of the volume of the cell (V_{cell}) and all bound beads (n*V_{MS}).

$${ρ}_{c o m p l e x} = \frac{{m}_{c e l l} + n ∗ {m}_{M P}}{{V}_{c e l l} + n ∗ {V}_{M P}}$$

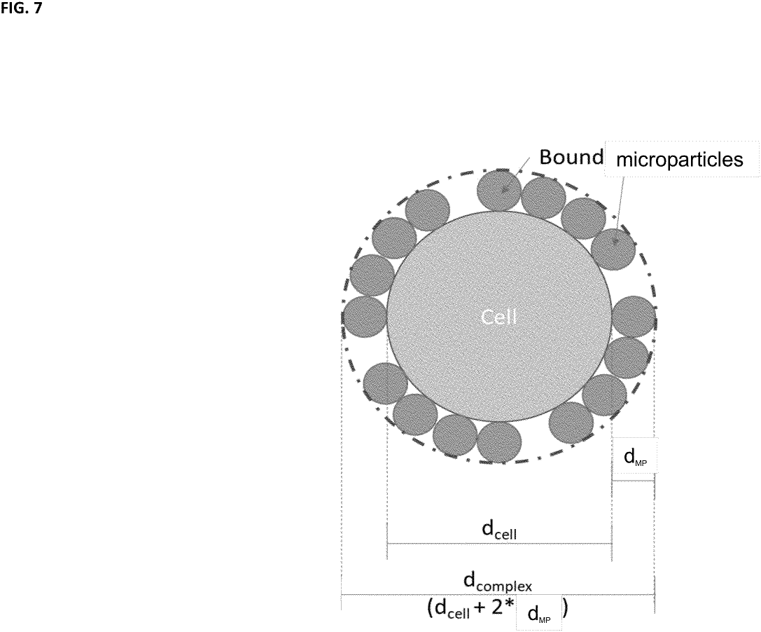

To estimate the theoretical maximum number of microparticles that can be bound to a cell, microparticles and cells are modeled as hard spheres forming a single layer of beads around the surface of the cell, as illustrated in FIG. 7. The volume available for the microparticles to occupy on the cell surface is determined by subtracting the volume of the cell from the volume of a sphere with diameter equal to twice the diameter of the microparticles (d_{MP}) plus the diameter of the cell (d_{cell}). The diameter of the complex is denoted d_{complex} in FIG. 7.

$${V}_{s h e l l} = {V}_{c o m p l e x} - {V}_{c e l l}$$

$${V}_{c e l l} = 4 / 3 π {\left(\frac{{d}_{c e l l}}{2}\right)}^{3}$$

$${V}_{c o m p l e x} = \frac{4}{3} π {\left(\frac{{d}_{c e l l} + 2 ∗ {d}_{M P}}{2}\right)}^{3}$$

$$M a x m i c r o p a r t i c l e s b o u n d = 0.64 ∗ \left(\frac{{V}_{s h e l l}}{{V}_{M P}}\right)$$

In the above calculation, the volume of the shell around the cell is multiplied by a spherical packing factor of 0.64 (random packing of equal spheres) and divided by the volume of a single microsphere to determine the number of microsphere that are able to bind to the cell. The packing factor assumes random packing of spheres. This may be a conservative estimate of how many spheres may be packed around a larger central sphere.

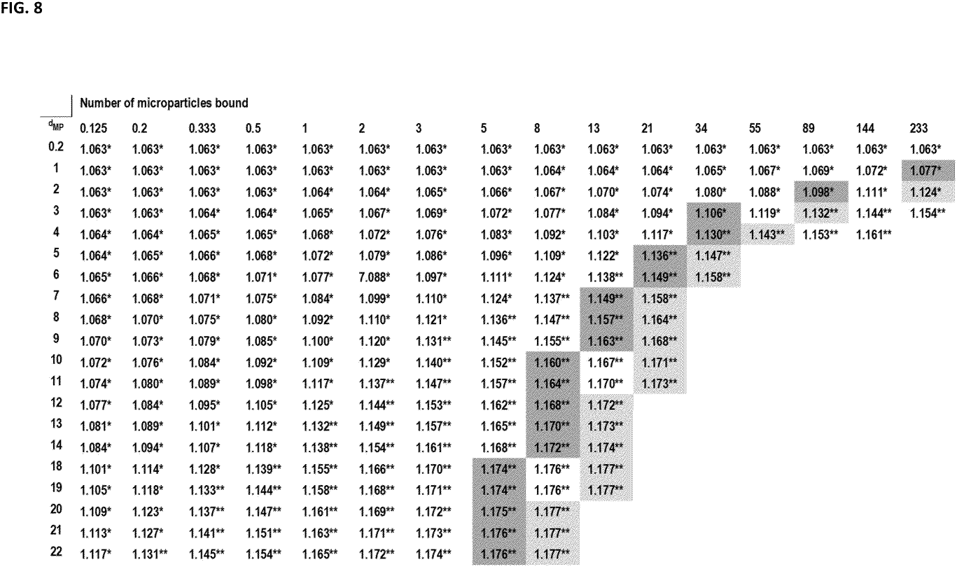

With a maximum number of microparticles that can fit around a cell established as an upper limit, estimates of microparticle-cell complex densities can be determined for various sizes of beads. For example, the table shown in FIG. 8 depicts the theoretical densities (g/cm³) of microparticle-cell complexes with various sizes of microparticles over a range of microparticle diameters and the numbers of microparticles bound per cell. The microparticles used in the calculation were assigned the density of 1.18 g/cm³. The cells used in the calculation were assigned the density of 1.063 g/cm³ and a diameter of 11.5 µm. In the table shown in FIG. 8, the table cells highlighted darker grey indicate a complex with about 40-60% microparticle coverage of the cell surface. The table cells highlighted in lighter grey indicate a complex with about 80-100% microparticle coverage of the cell surface. The complexes with densities less than or greater than 1.13 g/cm³ are labeled with single or double asterisks, respectively. The value of 1.13 g/cm³ was chosen for the specific calculation illustrated in FIG. 8, because it represented a cut-off density for the specific conditions of an exemplary magnetic levitation experiment. The complexes with the density below the cut-off would be collected in the bottom portion of the processing channel of the flowcell cartridge, and thus separated from the complexes with the densities above the cut-off, which would be levitating higher in the top portion of the processing channel of the flowcell cartridge. Other cut-off values may be used, depending on the particular experimental conditions and desired outcomes. The calculation illustrated in FIG. 8 estimates that, for larger microparticles, fewer microparticles per cell are needed to increase the density of the complex above the chosen cut-off, but there is also less available space to fit those larger microparticles on the cell surface. As determined by the calculation illustrated in FIG. 8, the microparticles with diameter of less 3 µm would not form a complex with the density above the chosen cut-off value, because it would require >100% coverage of the cell surface by the microparticles. Based on the illustrated calculation, the microparticles with the diameter of about 4-5 µm can achieve the density above the chosen cut-off value with about 50% coverage of the cell surface.

The calculation illustrated in FIG. 8 estimates the number of microparticles per cell in a complex to achieve a specific density. Given that the binding kinetics of a levitation-height altering agent or a magnetic agent are largely driven by the ligand-receptor binding interactions, a higher ratio of the microparticles to cells during complex formation (PTC ratio discussed elsewhere in the present disclosure) needed to achieve a target ratio of microparticles per cell in the complex. To satisfy equilibrium binding mechanics, steric hindrance, and diffusion of microparticles through solution to interact with the cells, the effective concentration of a linking agent included in the a levitation-height altering agent or a magnetic agent (which comprises a microparticle and a linking agent) needs to be maximized during complex formation, for example, by selecting the microparticles of the smaller diameter, in order to be able to increase the number of microparticles during complex formation. Another factor that needs to be taken into consideration is the product of the number of linking agent molecules bound to the surface of each microparticle and the total microparticles in suspension. For example, a microparticle with a mean diameter of 150 nm, 50% streptavidin coverage of each particle, and four binding sites per streptavidin molecule, would provide 1x10⁻¹⁴ µmol concentration of biotinylated antibody bound to streptavidin. For a sample of 2x10⁵ cells in 100 µl, a microparticle-to-cell ratio of 50,000:1 would yield about 1 µm concentration of the antibody, which is 10-100x the dissociation constant (K_{D}) of typical antibody interactions (10-100 nm). In comparison, a microparticle with a mean diameter of 5 µm at a microparticle-to-cell ratio of 40:1, with all the other conditions held the same, would still yield about 900 nm concentration of antibody. However, in this scenario, the amount of antibody attached to each microparticle is about 1000x as high. This limits access of the antibody to the cells during complex formation, as all the available antibodies are localized to a smaller total number of microparticles. It is envisioned that, for higher affinity linking agents, lower PTC ratios are required for effective complex formation, since the interaction between the linking agent and the cell is more likely to persist. Lower affinity linking agents likely require higher PTC ratios for effective complex formation to achieve and maintain the target microparticle-per-cell number in the complexes.

### Manufacture and Sourcing of Microparticles

Some methods of producing magnetic microparticles are described, for example, in Lu et al. “Modular and Integrated Systems for Nanoparticle and Microparticle Synthesis-A Review” Biosensors (2020) 10(11):165. Commercially available microparticles can also be used. Some exemplary magnetic microparticle sources are Nanopartz (Loveland, Colorado, USA), Biolegend (San Diego, California; for example, MojoSort™ nanobeads), BD Biosciences (San Jose, California; for example, BD™ IMag nanoparticles), Thermo Fisher Scientific (Waltham, Massachusetts; for example, Dynabeads®), Creative Diagnostics (New York, New York, USA), Spherotech (Lake Forest, Illinois, USA), Bangs Laboratories (Fishers, Indiana, USA), Miltenyi Biotec (Bergisch Gladbach, North Rhine-Westphalia, Germany; for example, MACS® MicroBeads), Bio-Techne (Minneapolis, Minnesota), Bioclone (San Diego, California; for example, BcMag beads), Polysciences (Warrington, Pennsylvania, USA), and STEMCELL Technologies (Vancouver, Canada; for example, RapidSphere™ microbeads).

### Linking Agent

A “linking agent” is used to couple a magnetic microparticle(s) to a component of interest (e.g., a cell of interest, organelle, nucleic acids). A linking agent specifically binds to the cell or other analyte. A linking agent may include a specific binding molecule or molecules, examples of which are discussed in more detail elsewhere in the present disclosure. One example of a linking agent comprises an antibody that specifically binds to a cell surface protein displayed on a cell of interest. Other types of linking agents comprise aptamers, ligands (that are bound by a cell-surface receptor), including, but not limited to, small molecule ligands and polypeptide or protein ligands, lipophilic tags, and nucleic acids (at least a portion of which is complementary to a target nucleic acid). For example, removal of mRNA from a sample can be performed by coupling an Oligo-dT to magnetic microparticles, which then bind to the poly-A tail at the end of mRNA. In another example, total RNA can be removed from a sample by coupling random hexamer oligonucleotides to magnetic microparticles, which then bind to random RNA hexanucleotides.

### Antibody

The term “antibody” and the related terms, in the broadest sense, are used in the present disclosure to denote any product, composition or molecule that contains at least one epitope binding site, meaning a molecule capable of specifically binding an “epitope” - a region or structure within an antigen. The term “antibody” encompasses whole immunoglobulin (i.e., an intact antibody) of any class, including natural, nature-based, modified, and non-natural (engineered) antibodies, as well as their fragments. The term “antibody” encompasses “polyclonal antibodies,” which react against the same antigen, but may bind to different epitopes within the antigen, as well as “monoclonal antibodies” (“mAbs”), meaning a substantially homogenous population of antibodies or an antibody obtained from a substantially homogeneous population of antibodies. The antigen binding sites of the individual antibodies comprising the population of mAbs are comprised of polypeptide regions similar (although not necessarily identical) in sequence. The term “antibody” also encompasses fragments, variants, modified and engineered antibodies, such as those artificially produced (“engineered”), for example, by recombinant techniques. For instance, the term “antibody” encompasses, but is not limited to, chimeric antibodies and hybrid antibodies, antibodies with dual or multiple antigen or epitope specificities, and fragments, such as F(ab')2, Fab', Fab, hybrid fragment, single chain variable fragments (scFv), “third generation” (3G) fragments, fusion proteins, single domain and “miniaturized” antibody molecules, and “nanobodies.”

### Aptamer

Nucleic acid aptamers are RNA or single stranded DNA molecules, which can fold into various architectures and bind to a wide array of targets including other nucleotides or proteins. For example, aptamers to tumor cell-surface markers, including HER-2, a breast cancer cell surface marker, may be used for isolating or removing tumor cells from a tissue sample. Aptamers Targeting Tumor Cell-Surface Protein Biomarkers, as well as selection of such aptamers, are discussed, for example, in Mercier et al., Cancers (Basel) 9(6):69 (2017) doi: 10.3390/cancers9060069.

### Lipophilic Tag

The linking agent may be a lipophilic tag. Lipophilic tags are lipophilic molecules that can associate with and/or insert into lipid membranes such as cell membranes and organelle membranes. Examples of lipophilic molecules include sterol lipids (e.g., cholesterol or tocopherol), steryl lipids, lignoceric acid, and palmitic acid. By themselves, lipophilic tags do not accomplish specific binding. However, they may be used to specifically target cell membranes in the mixtures of cells with other particles. In addition, different samples can be tagged with differentially-labeled lipophilic tags.

### Other Specific Binding Partners

The expression “specific binding molecule” denotes a molecule capable of specifically or selectively binding another molecule or a region or structure within another molecule, which may be termed “target,” “ligand” or “binding partner.” The terms “specific binding,” “selective binding” or related terms refer to a binding reaction in which, under designated conditions, a specific binding molecule or a composition containing it binds to its binding partner or partners and does not bind in a significant amount to anything else. Binding to anything else other than the binding partner is typically referred to as “nonspecific binding” or “background.” The absence of binding in a significant amount is considered, for example, to be binding less than 1.5 times background (i.e., the level of non-specific binding or slightly above non-specific binding levels). Some non-limiting examples of specific binding are antibody-antigen or antibody-epitope binding, binding of oligo- or polynucleotides to other oligo- or polynucleotides, binding of oligo- or polynucleotides to proteins or polypeptides (and vice versa), binding or proteins to polypeptides other proteins or polypeptides, receptor-ligand binding, and carbohydrate-lectin binding. Accordingly, specific binding molecules can be or can include a protein, a polypeptide, an antibody, an oligo- or polynucleotide, a receptor, or a ligand. Specific binding molecules can be natural or engineered. For example, both engineered and naturally occurring nucleic acid or peptide aptamers can serve as specific binding molecules in the embodiments of the present invention. This list is not intended to be limiting, and other types of specific binding molecules may be employed. The term “target molecule” is used to denote a molecule or a part thereof, including a biological molecule (such as, but not limited to, a protein, a peptide, lipid, a nucleic acid, a fatty acid, or a carbohydrate molecule, such as an oligosaccharide), or a nonbiological molecule (including a small molecule, such a small molecule drug or a small molecule ligand). A specific binding molecule, such as antibody, specifically binds to the target molecule.

### Indirect Binding

Specific binding molecules, such as, but not limited to, antibodies, can be directly attached to magnetic microparticles, for example, by surface conjugation, coating, or adsorption. However, specific binding molecules need not be directly attached to magnetic microparticles, and can be used for complexing magnetic microparticles with a target cell via an intermediary non-covalent binding interaction. For example, specific binding molecule is a biotinylated antibody capable of specifically binding to a target cell, and density-modifying microparticles are coated with avidin, streptavidin, neutravidin, or any form of modified avidin, which can be referred to as “avidin-like compound.” An intermediary binding interaction between an avidin-like compound on the magnetic microparticles and biotin moiety of the antibody allows for formation of a complex between a target cell and a density-modifying microparticle. In another example, specific binding molecule is an antibody capable of specifically binding to a target cell (“primary antibody”), and magnetic microparticles are coated with protein A, protein S, or an anti-antibody (that is, an antibody against primary antibody). An intermediary binding interaction between protein A, protein S, or an anti-antibody on the magnetic microparticles and the primary antibody allows for formation of a complex between a target cell and a magnetic microparticle.

### Fluidics

As used in the present disclosure, the term “fluidic” refers to a system, device or element for handling, processing, ejecting and/or analyzing a fluid sample including at least one “channel” as defined elsewhere in the present disclosure. The term “fluidic” includes, but is not limited to, microfluidic and nanofluidic.

As used in the present disclosure, the terms “channel”, “flow channel,” “fluid channel” and “fluidic channel” are used interchangeably and refer to a pathway on a fluidic device in which a fluid can flow. Channel includes pathways with a maximum height dimension of about 100 mM, about 50 mM, about 30 mM, about 25 mM , about 20 mM, about 15 mM , about 10 mM , about 5 mM , about 3 mM , about 2 mM , about 1 mM , or about 0.5 mM. For example, the channel between magnets can have cross-sectional dimensions (height by width) of about 10 mM x 10 mM , about 10 mM x5 mM , about 10 mM x 3 mM , about 10 mM x 2 mM , about 10 mM x 1 mM , or about, about 10 mM x0.5 mM , about 5 mM x 10 mM , about 5 mM x5 mM , about 5 mM x 3 mM , about 5 mM x 2 mM , about 5 mM x 1 mM , about 5 mM x 0.5 mM, about 3 mM x 10 mM , about 3 mM x5 mM , about 3 mM x 3 mM , about 3 mM x 2 mM , about 3 mM x 1 mM , about 3 mM x0.5 mM , about 2 mM x 10 mM , about 2 mM x5 mM , about 2 mM x 3 mM , about 2 mM x 2 mM , about 2 mM x 1 mM , about 2 mM x0.5 mM , about 1 mM x 10 mM , about 1 mM x5 mM , about 1 mM x 3 mM , about 1 mM x 2 mM , about 1 mM x 1 mM , about 1 mM x0.5 mM , about 0.5 mM x 10 mM , about 0.5 mM x5 mM , about 0.5 mM x 3 mM , about 0.5 mM x 2 mM , about 0.5 mM x 1 mM , or about 0.5 mM x 0.5 mM. The internal height of the channel may not be uniform across its cross-section, and geometrically the cross-section may be any shape, including round, square, oval, rectangular, or hexagonal. The cross-section may vary along the length of the channel. The term “channel” includes, but is not limited to, microchannels and nanochannels, and, with respect to any reference to a channel in the present disclosure, such channel may comprise a microchannel or a nanochannel.

As used in the present disclosure, the term “fluidically coupled” or “fluidic communication” means that a fluid can flow between two components that are so coupled or in communication.

### Magnetic Levitation

The expression “magnetic levitation,” in the context of the present disclosure and as described, for example, in U.S. Pat. Application No. US 14/407,736, generally involves subjecting diamagnetic, paramagnetic, ferromagnetic, ferrimagnetic or antiferromagnetic materials or “objects” suspended in a paramagnetic fluid medium to a magnetic field, such as a magnetic field gradient that forms between two magnets. The magnetic field generates a nonuniform pressure equivalent to the magnetic energy density in the paramagnetic fluid medium. In a magnetic field gradient, the objects appear to be repelled from the regions of high magnetic field. In actuality, the object is displaced by an equal volume of the paramagnetic fluid medium. The attractive interaction between the paramagnetic fluid medium and the regions of high magnetic field can result in the “levitation” of the object. The “levitation height” of an object, in the two-magnet setup, can be defined as desired. For example, in certain embodiments, “levitation height” can be defined as the distance between the center of the levitating object and the top surface of the bottom magnet, but any desired reference point can be utilized. By applying the magnetic field in such a manner that the force on the objects is opposed by another uniform force (e.g., the force of gravity), a balance is achieved for the object that is directly related to its density.

### Paramagnetic Fluid Medium

In the context of the present disclosure, a “paramagnetic fluid medium” may comprise a paramagnetic material and a solvent. In some embodiments of the methods described in the present disclosure, the paramagnetic fluid medium is biocompatible, i.e. capable of being mixed with live cells and not impact the viability of the cells or impacting cellular behavior. A paramagnetic material may include one or more of: gadolinium, titanium, vanadium, dysprosium, chromium, manganese, iron, nickel, gallium, including their ions. For example, a paramagnetic material may include one or more of the following ions: titanium (III) ion, gadolinium (III) ion, vanadium (I) ion, nickel (II) ion, chromium (III) ion, vanadium (III) ion, dysprosium (III) ion, cobalt (II) ion, and gallium (III) ion. In some embodiments, a paramagnetic material comprises a chelated compound, such as, but not limited to, a gadolinium chelate, a dysprosium chelate, or a manganese chelate. In some examples, a paramagnetic material may comprise one or more of [Aliq]₂ [MnCl₄], [Aliq]₃ [GdCl₆], [Aliq]₃ [HoCl₆], [Aliq]₃ [HoBr₆], [BMIM]₃ [HoCl₆], [BMIM] [FeCl₄], [BMIM]₂ [MnCl₄], [BMIM]₃ [DyCl₆], BDMIM]3 [DyCl₆], [AlaC1] [FeCl4], [AlaCl]₂ [MnCl₄], [AlaCl]₃ [GdCl₆], [AlaCl]₃ [HoCl₆], [AlaCl]₃ [DyCl₆], [GlyC2] [FeCl₄]. In one exemplary embodiment, a paramagnetic material is gadobutrol. A paramagnetic material may be present in the paramagnetic fluid medium at a concentration of at least about 10 mM , 20 mM , 30 mM , 40 mM , 50 nm, 60 mM , 70 mM , 80 mM , 90 mM , 100 mM , 120 mM , 150 mM , 200 mM , 250 mM , 300 mM, 500 mM, 1 M, about 10 mM to about 50 mM , about 25 mM to about 75 mM , about 50 mM to about 100 mM , about 100 mM to about 150 mM , about 150 mM to about 200 mM , about 200 mM to about 250 mM , about 250 mM to about 300 mM, about 300 mM to about 500 mM, or about 500 mM to about 1 M. In an exemplary embodiment, a paramagnetic material comprises gadolinium, and the paramagnetic material is present in the paramagnetic fluid medium at a concentration of at least about 10 mM , 20 mM , 30 mM , 40 mM , 50 mM , 60 mM , 70 mM , 80 mM , 90 mM , 100 mM , 200 mM , 300 mM, 400 mM, 500 mM, 600 mM, 700 mM, 800 mM, 900 mM, about 10 mM to about 50 mM , about 25 mM to about 75 mM , about 50 mM to about 100 mM , about 50 mM to about 200 mM , about 50 mM to about 300 mM, about 50 mM to about 400 mM, about 50 mM to about 500 mM, about 50 mM to about 600 mM, about 50 mM to about 700 mM, about 50 mM to about 800 mM, about 50 mM to about 900 mM, or about 50 mM to about 1 M. It is understood that, in addition to a paramagnetic material and a solvent, a paramagnetic fluid medium may comprise other components, such as salts or additives, for example, but not limited to, additives that function to maintain cellular integrity.

### Magnetic Levitation Systems and Components

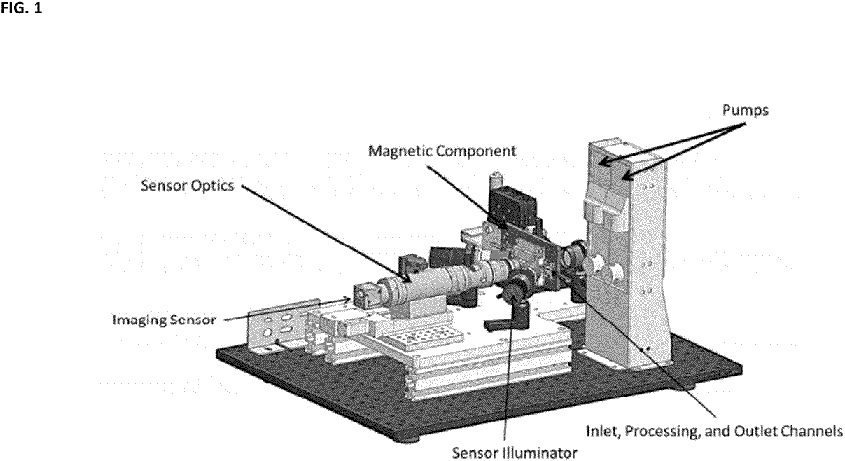

Exemplary magnetic levitation systems that and their components are described, for example, in Durmus et al. and in U.S. Pat. Application No. 17/449,438, filed Sep. 29, 2021. While various embodiments of the invention provided in the present disclosure are not limited by any particular magnetic levitation system, a brief description is included to facilitate the understanding of the methods, kits and systems according to the embodiments of the present invention. An exemplary magnetic levitation system is schematically illustrated in FIG. 1.

### Flowcell Cartridge

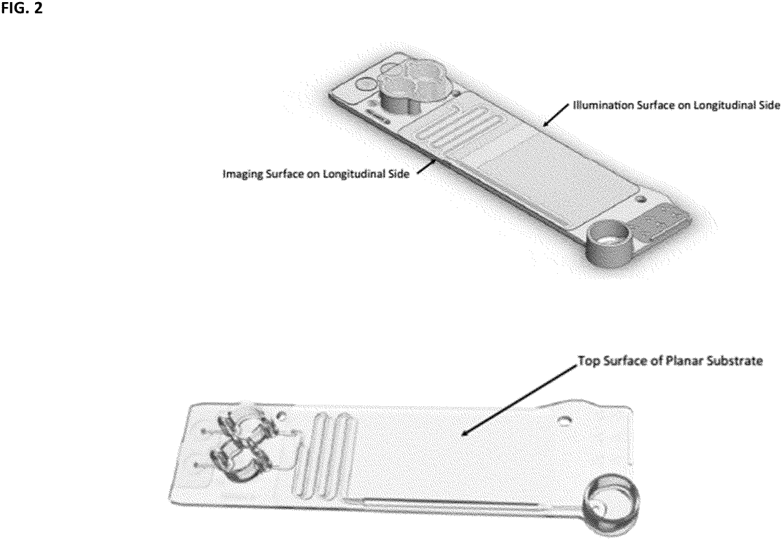

At least some methods and kits according to the embodiments of the present invention involve a flowcell cartridge for use in a magnetic levitation system. An exemplary flowcell cartridge is schematically illustrated in FIG. 2. An exemplary flowcell cartridge may include a planar substrate comprising an upper surface and a lower surface, a first longitudinal side forming an imaging surface, a second longitudinal side forming an illumination surface, and a first and second transverse side, an inlet well on an upper surface, an inlet channel, a sample processing channel in fluidic communication with the inlet channel and positioned substantially parallel to a longitudinal side, a sample splitter within the processing channel, a plurality of outlet channels in fluidic communication with the processing channel, and a plurality of collection wells in fluidic communication with each of the plurality of outlet channels. The planar configuration allows for all required flowcell cartridge functions to be integrated into the flowcell cartridge and increases performance and reproducibility in a laboratory or clinical setting. In operation, it is important for enhanced performance that the flow the processing channel and into the outlet channel be as free of turbulence as possible. The processing channel may be offset within the plane of the of the planar substrate to be spatially biased to the imaging surface.

The flowcell cartridge can be formed by injection molding, etching, laser ablation, machining, or 3D printing. When imaging within the flowcell cartridge is desired, the planar substrate comprises an optically transparent material. Glass, plastic, or polymer materials including cyclic olefin polymer (COP) or cyclic olefin copolymer (COC) are some examples of suitable optically transparent materials. Dimensions of the planar substrate can be at least 50 mM in length, 20 mM in width, and at least 1.5 mM in thickness. Optional ranges are at least 100 mM in length, 35 mM in width, and about 2 to about 6 mM in thickness. The longitudinal sides of the cartridge can act as waveguides for illumination and imaging. For that reason, the processing channel is offset in the plane of the substrate and is parallel and adjacent to the imaging longitudinal side of the substrate. Distances from the imaging side wall can be from about 0.5 mM to about 10 mM , preferably from about 0.5 mM to about 5 mM , optionally from about 1 mM to about 3.5 mM. In an embodiment the processing channel spacing from the imaging wall is about 2 mM . The volume of the processing channel can be from about 10 µL to about 800 µL, from about 50 µL to about 600 µL, 100 µL to about 400 µL, about 150 µL to about 300 µL, at least about 150 µL, at least about 200 µL, at least about 250 µL, or at least about 300 µL. The combined volume of the outlet channels can be greater than the volume of the processing channel. The flow volume split between two outlet channels can be an even split or can range from about 4:1 to about 1:4, about 3:1 to about 1:3 or about 2:1 to about 1:2 or can vary from 1:1 by about 50% or less, or about 40% or less, or about 30% or less, or about 15% or less when in operation in the system embodiments.

A flowcell cartridge optionally includes collection wells on the planar substrate. The collection wells feature an inlet that is in fluidic communication with the outlet channel. The inlet can be at a first well height and configured with a step transitioning from the inlet port aperture to the floor of the well. This provides a transition surface for the flow of sample fraction into the well and can inhibit back siphoning of the sample fraction into the outlet channel as well as bubble formation within the collection well. An outlet channel within the collection well can be provided with an opening that is at a height off the floor of the collection well that is higher than the opening of the inlet channel. The internal outlet can be placed in communication with a flow modulator. In some instances, the flow modulator is an individual pump to provide flow through the flowcell cartridge. In operation, the collection well is sealed with a layer of material or film to provide an enclosed system to allow flow or pumping of sample and sample fractions through the flowcell cartridge. In assembling the flowcell cartridge layers, and when an adhesive is used, a biocompatible adhesive can be used for biological applications. Correct adhesive selection is necessary to minimize or prevent leaching of adhesive components into the solution, adhering to cells or binding molecules from solution, being autofluorescent, having texture which increases the surface area and hence the impact on cells, and overly hydrophilic or hydrophobic. An example of a suitable adhesive is a silicone or silicone-based adhesive.

### Separation System

An exemplary magnetic levitation-based separation system (illustrated in FIG. 1) comprises a receiving block for retaining a flowcell cartridge, an optical system comprising an optical sensor, a lens, and an illumination source, and plurality of flow modulation components. The receiving block removably places the flowcell cartridge in optical alignment with the optical system, removably engages a magnetic component adjacent to the processing channel of the flowcell cartridge, and removably places a plurality of outlet channels of the flowcell cartridge in fluidic communication with the plurality of flow modulation components. The optical system may be constructed to provide microscopic imaging of the processing channel of the flowcell cartridge. Optionally, the optical system may be constructed and arranged to provide imaging for florescence emission with optional ultraviolet light exciter modules. The optical system may comprise a source of visible optical illumination constructed and arranged to provide light transmission through the processing channel within the planar substrate. The receiving block is constructed and arranged to hold the planar flowcell cartridge in an orientation to the optical system, such that the imaging optics are aligned with the imaging side of the planar cartridge and the visible light emitter is in an orientation to illuminate the illumination side of the planar flowcell cartridge. Optionally, the optical system can further comprise one or more sources of ultraviolet or visible illumination constructed and arranged to place the illumination in an angular orientation the imaging side of the planar cartridge to excite fluorophores within the processing channel for the cartridge. For imaging of fluorescent entities internal to the processing channel optical system optionally comprises a dual bandpass filter preferably passing emitted radiation in bands centered at wavelengths at about 524 nm and 628 nm.

An optional feature of the receiving block is a series of flow modulator adapters that interface with outlets on the top or bottom of the flowcell cartridge. The adapters facilitate fluidic communication with flow modulators, such as a pump in the system, with outlet channels of the flow cells such as the collection well outlet channels. Once the flowcell cartridge is inserted into the receiving block, the receiving block is mechanically actuated to support the cartridge, aligning the illumination and imaging sides of the planar cartridge with the optical imaging system, aligning the magnetic components to position them above and below the flowcell processing channel, and, where desired, place the flow modulator adapters in fluidic communication with corresponding outlet channels of the flowcell cartridge. The flow modulators of the system provide flow to the sample and sample fractions within the flowcell cartridge. The flow rate provided by the flow modulators can range from as low as 1 µL per minute to as high as 1 mL per minute during separations. The flow rate can be at or at least about 25 µL per minute, at or at least about 50 µL per minute, at or at least about 100 µL per minute, at or at least about 200 µL per minute, at or at least about 250 µL per minute, at or at least about 300 µL per minute, or from about 300 µL per minute to about 1 mL per minute. The total sample volume flowrate can be about 50 µL/min, about 75 µL/min, about 100 µL/min, about 150 µL/min, about 200 µL/min or about 300 µL/min. The flow volume split between two outlet channels can be an even split or can range from about 4:1 to about 1:4, about 3:1 to about 1:3 or about 2:1 to about 1:2 or can vary from 1:1 by about 50% or less, or about 40% or less, or about 30% or less, or about 15% or less when in operation in the system embodiments.

A magnetic levitation system is capable of magnetically levitating particles suspended in a paramagnetic fluid medium within a processing channel or inlet channel of a flowcell cartridge. The interaction of the magnetic field with the paramagnetic properties of particles within a paramagnetic fluid medium can either provide a repulsive or attractive effect on the particles to facilitate their separation or concentration. The magnetic field in a magnetic fluid medium is created by magnets, which can be permanent magnets or electromagnets. The maximum energy product of magnets can range from about 1 Mega-Gauss Oersted to about 1000 Mega-Gauss Oersted, or from about 10 Mega-Gauss Oersted to about 100 Mega-Gauss Oersted. The surface field strength of magnets can range from about 0.01 Tesla to about 100 Tesla, or from about 1 Tesla to about 10 Tesla. The remanence of magnets can range from about 0.5 Tesla to about 5 Tesla, or from about 1 Tesla to about 3 Tesla. Magnets can be made from a material comprising neodymium alloys with iron and boron, neodymium, alloys of aluminum with nickel, neodymium alloys with iron, aluminum and cobalt alloyed with iron, samarium-cobalt, other alloys of rare earth elements with iron, alloys of rare earth alloys with nickel, ferrite, or combinations thereof. When a magnetic levitation system comprises a plurality of magnets, magnets can be made from the same material or are made from different materials.

An asymmetric magnetic field can be achieved by using a stronger magnetic material on one side of a fluidic channel of a flowcell cartridge and a weaker magnetic material on the opposite side of the fluidic channel of a flowcell cartridge. An asymmetric magnetic field can be achieved by positioning a magnet closer on one side of a channel than a magnet on the other side. An asymmetric magnetic field can be achieved by using a magnetic material on one side of a fluidic channel of a flowcell cartridge and a substantially similar magnetic material on the opposite side of the fluidic channel of a flowcell cartridge. An upper magnet and a lower magnet may be substantially the same size. In one example, the upper magnet may comprise neodymium, the lower magnet may comprise samarium-cobalt. Alternatively, the upper magnet may comprise samarium-cobalt, and the lower magnet may comprise neodymium. Alternative magnet configurations may be used. A magnetic levitation system may include multiple upper magnets and multiple lower magnets positioned around a fluidic channel of a flowcell cartridge. In one example, upper magnets may include an anterior upper magnet, a central upper magnet, and a posterior upper magnet. Lower magnets may include an anterior lower magnet, a central lower magnet, and a posterior lower magnet. In another example, a magnetic levitation system may include an anterior upper magnet, a posterior upper magnet, an anterior lower magnet, and a posterior lower magnet, with the magnets positioned around a fluidic channel of a flowcell cartridge, and the anterior upper magnet and the posterior lower magnet are positioned in a magnetic repelling orientation. Exemplary NdFeB magnetic component dimensions include, for a bottom magnet component about 50 x 15 x 2 mM (magnetized through the 15 mM axis), 50 x 5 x 2 mM (magnetized through the 5 mM axis) for a top magnet component. Other exemplary magnet dimensions include 60 x 15 x 2 mM , 60 x 5 x 2 mM , 75 x 10 x 3 mM , 75 x 20 x 3 mM , and 25 x 15 x 2 mM . An exemplary magnet configuration of a magnetic levitation system includes an upper and lower magnet with dimensions of about 75 x 20 x 3.2 mM, and a spacing between upper and lower magnets of about 2.5 mM, about 3.0 mM, about 3.5 mM, about 2.9 mM, about, 3.0 mM, about 3.1 mM, about 3.2 mM, about 3.3 mM, or about 2.72 mM, about 2.88 mM, about 2.98 mM, about 3.18 mM, about 3.20 mM, or about 3.37 mM. One more exemplary magnet configuration of a magnetic levitation system has an upper magnet and a lower magnet, with the lower magnet extending into an inlet channel of a flowcell cartridge. The bottom magnet dimensions can be about 50 mM to about 100 mM x about 10 mM to about 30 mM x about 2 mM to about 4 mM.

### Samples

The terms “sample” or “samples,” and the related terms and expressions, as used in the present disclosure, are not intended to be limiting, unless qualified otherwise. These terms refer to any product, composition, cell, tissue or organism. Generally, the terms “sample” or “samples” are not intended to be limited by their source, origin, manner of procurement, treatment, processing, storage or analysis, or any modification. Some examples of the samples are solutions, suspensions, supernatants, precipitates, or pellets. Samples can contain or be predominantly composed of cells or tissues, or can be prepared from cells or tissues. However, samples need not contain cells. Samples may be mixtures of or contain biological molecules, such as nucleic acids, polypeptides, proteins (including antibodies), lipids, carbohydrates etc. Samples may be biological samples. For example, a “sample” may be any cell or tissue sample or extract originating from cells, tissues or subjects, and include samples of animal cells or tissues as well as cells of non-animal origin, including plant and bacterial samples. A sample can be directly obtained from an organism, or propagated, or cultured. Some exemplary samples are cell extracts (for examples, cell lysates), suspensions of cell nuclei, liquid cell cultures, cell suspensions, biological fluids (including, but not limited to, blood, serum, plasma, saliva, urine, cerebrospinal fluid, amniotic fluid, tears, lavage fluid from lungs, or interstitial fluid), tissue sections, including needle biopsies, microscopy slides, frozen tissue sections, or fixed cell and tissue samples.

### Exemplary Cell Types

Cells tagged with levitation height-altering and/or magnetic agents may be, for illustration and not limitation, human cells, non-human animal cells, plant cells, eukaryotic cells, prokaryotic cells, etc. For example, tagged cells may be, but are not limited to, human or non-human immune cells, endothelial cells, and T-cells. Tagged cells may be in a heterologous population of untagged cells, which may be, for illustration and not limitation, human cells, non-human animal cells, plant cells, eukaryotic cells, prokaryotic cells, etc. Tagged cells may be from different cell lineages, or could be the same cell lineage but different in activation state, differentiation state, or some other property.

### Tagging Cells

One or more than one (2, 3, 4, or more than 4) cell types may be processed in a single separation step. When two or more cell types are tagged, each cell type may be tagged with a different levitation height-altering and/or magnetic agent. Alternatively two or more different cell types may be tagged with the same levitation height-altering and/or magnetic agent. For example, two or more different cell types may have the same surface markers, and levitation height-altering and/or magnetic agent comprising the same linking agent may therefore bind to both cell types. In another example, a levitation height-altering and/or magnetic agent comprises two or more different linking agents that can bind to respective two or more surface markers. In this situation, the levitation height-altering and/or magnetic agent can bind to two or more different cell types with different surface markers corresponding to two or more different linking agents. In some embodiments, two or more different cell types may be tagged with different levitation-height altering agents that have the same levitation-height altering properties.

### Tagging Process

As a part of a process of tagging cells, magnetic microparticles comprising a linking agent specific to a target cell type (which, together, may be referred to as a “levitation-height altering agent” or “magnetic agent”) are mixed with the cells. A ratio of the magnetic particles to cells in the mixture (PTC ratio, discussed elsewhere in the present disclosure) is optimized based on various parameters. One of these parameters may be the target cell type, which determines its levitation profile, but may also affect the number of levitation height-altering and/or magnetic agent units with which each cell may complex. For example, if the linking agent is specific for cell surface markers, the number of markers on a particular target cell type will determine how many units of the levitation height-altering and/or magnetic agent can bind to this cell. Another parameter may be the affinity of the linking agent, such as an antibody, for a particular cell type. One more parameter is the magnetic field strength applied during magnetic levitation. Other parameters that may be taken into account when determining PTC ratio may be magnetic susceptibility of the microparticles included in the levitation height-altering and/or magnetic agent, microparticle size, and/or microparticle density. It is understood that other parameters, not listed here, may also be taken into account, and also that PTC ratio may be experimentally determined and/or optimized for a particular tagging and/or separation application. Non-limiting PTC ratios used in the embodiments of the methods described in the present disclosure may be (but are not limited to) from about 1 to about 100,000, from about 1 to about 50,000, from about 1 to about 10,000, from about 1 to about 1,000, from about 1 to about 100, from about 10,000 to about 100,000, from about 10,000 to about 50,000, or from about 50,000 to about 100,000, When multiple cell types are present, magnetic microparticles with different linking agents specific for each cell type may be mixed together prior to addition to cell mixture, or magnetic microparticles with different linking agents and cells will be mixed together in one step. In some cases, the tagging process may be performed serially on a cell mixture, mean that microparticles with different linking agents specific for each cell type may be applied after each tagging and separation step.

### Methods of Cell Separation

Described in the present disclosure and included among the embodiments of the present invention are improved methods (processes) of cell separation by magnetic levitation. Such methods may also be referred to as “cell separation methods,” “methods of separating cells,” “methods of cell isolation,” “cell isolation methods,” “methods of isolating cells,” “cell concentration methods,” “methods of concentrating cells,” “cell segregation methods,” “methods of segregating cells,” and by other related terms and expressions, which are not intended to be limiting.

The methods described in the present disclosure are useful for separating one or more types of cells from a population of cells including multiple cell types. Such multiple cell types may include animal cells, including human cells and non-human animal cells, mixtures of human and non-human cells, plant cells, as well as cells of other origins, including, but not limited to, bacterial cells, protozoan cells, algal cells, etc. Multiple cell types may include dead cells, living cells, healthy cells, pathological cells, infected cells, transfected cells, or genetically modified cells. Cells separated according to the methods of the present disclosure may include cells in various states (for example, stem cells, differentiated cells, etc.). Cells separated according to the methods of the present disclosure can be directly obtained from an organism (or be an organism itself), or propagated or cultured. Cells can be subject to various treatments, storage or processing procedures before being separated according to the methods described in the present disclosure. Generally, the terms “cell,” “cells,” “cell type” (or the related terms and expressions) are not intended to be limited by their source, origin, manner of procurement, treatment, processing, storage or analysis, or any modification. Some non-limiting examples of the cell types that may be suitable for being separated by the methods described in the present disclosure are macrophages, alveolar type II (ATII) cells, stem cells, adipocytes, cardiomyocytes, embryonic cells, tumor cells, lymphocytes, red blood cells (erythrocytes), epithelial cells, ova (egg cells), sperm cells, T cells, B cells, myeloid cells, immune cells, hepatocytes, endothelial cells, stromal cells, and bacterial cells. A population of cells that includes multiple cell types may be derived from various types of samples, which are discussed elsewhere in the present disclosure.

Cell separation methods according to some of the embodiments of the present invention involve performing binding of levitation height-altering and/or magnetic agent or agents to a cell of a particular type or types (which can be referred to as “target cell” or “target cell type”) found in a population of cells comprising multiple cell types. Such binding can also be described as “forming a complex,” “complex formation” or “complexing.” Preferential binding of levitation height-altering and/or magnetic agent to a target cell is accomplished by using linking agents comprising binding molecules capable of specifically or selectively binding the target cell. Such molecules can be called “specific binding molecules.” An example of a specific binding molecule is an antibody specific against a cell surface marker or a molecule specific for a target cell type. Some examples of surface markers are CD45, CD3, CD4, CD8, CD19, CD40, CD56, CD11b, CD14, CD15, EpCAM, ICAM, CD235, HER-2, HER-3, CD66e, Integrins, E- P- L-Selectins, EGFR, EGFRVIII, PDGFR β, c-MET, MUC-1, OX-40, CD28, CD133, CD30 TNFRSF8, CTLA4, CD71, CD16α VCAM-1, Nucleolin, and Myelin Basic Protein. It is to be understood that different cells may have different surface markers. For example, human and non-human animal cells, such as mouse cells, may have different surface markers. Embodiments of the cell separation methods of the present invention may utilize more than one (one or more, two or more, three or more, four or more, etc.) specific binding molecule. In other words, embodiments of the cell separation methods of the present invention may utilize multiple specific binding molecules capable of forming complexes with different target cell types in a population of cells containing multiple cell types.

In the embodiments of the cell separation methods that involve binding of a levitation height-altering and/or magnetic agent to a cell of a particular type or types, the binding can be accomplished by various steps. For example, the binding can be accomplished by contacting, combining or incubating a levitation height-altering agent comprising a linking agent comprising a specific binding molecule with a population of cells comprising multiple cell types, potentially including a target cell type, under conditions in which the magnetic (for example, paramagnetic or superparamagnetic) microparticle binds individual cells of the target cell type to form complexes, each complex one or more microparticles bound to a cell of the target cell type. However, embodiments of the cell separation methods according to the present invention need not include any steps related to binding of a levitation height-altering and/or magnetic agent to cells. Complexes of levitation height-altering and/or magnetic agent and target cells can be formed before the start of the method and be provided at the beginning of the cell separation methods according to the embodiments of the present invention. In other words, the method can start with a step of providing a complex of a levitation height-altering and/or magnetic agent and a cell of a target cell type (or target cell), optionally included in a population of cells comprising multiple cell types.

Some embodiments of cell separation methods according to the present invention include a step or steps related to forming a suspension, in a paramagnetic fluid medium, of a complex of one or more levitation height-altering and/or magnetic agent and a cell of a target cell type, and a plurality of the cells of the multiple cell types. In some embodiments, such a suspension may be provided at the start of the method. Cell separation methods according to the embodiments of the present invention involve introducing the suspension into a processing channel of a flowcell cartridge of a magnetic levitation system. The flowcell cartridge comprises at least one outlet channel, and a processing channel having a length and a vertical height. Cell separation methods according to the embodiments of the present invention involve exposing the processing channel to a magnetic field for a period of time sufficient for at least some of the complexes (or at least one complex) of one or more levitation height-altering and/or magnetic agents and a cell of a target cell type to separate from the cells of the multiple cell types not bound to levitation height-altering and/or magnetic agent or agents, thereby forming a first portion of the suspension enriched with the complex relative to the suspension, and a second portion of the suspension depleted of the complex relative to the suspension. The exposure to the magnetic field can be performed in a stop-flow mode or continuous flow mode of the flowcell cartridge.

In some embodiments of the cell separation methods, a vertical position of the flowcell cartridge in the magnetic field is changeable, which may affect levitation height of the complexes (or at least one complex) of one or more levitation-height altering agent and a cell of a target cell type relative to magnets of the magnetic levitation system. Changing a vertical position of the flowcell cartridge may be advantageously used to access different portions of the suspension. In some embodiments of the cell separation methods, the composition of the paramagnetic fluid can be adjusted to improve cell separation. For example, the concentration of the paramagnetic compound changes the physical space occupied by a range of densities, such that it is possible to target a specific range of densities within the separation channel by adjusting the concentration of the paramagnetic compound. With the increase in the concentration of the paramagnetic compound in the paramagnetic fluid, the range of the particle densities that can be levitated between the magnets becomes broader. However, if the magnet spacing is not adjusted in this scenario, the physical separation distance between the particles of different densities becomes smaller. Conversely, with the decrease of the concentration of the paramagnetic compound in the paramagnetic fluid, the range of the particle densities that can be levitated between the magnets narrows, but the physical separations distance between the particles of different densities increases. Accordingly, in the methods of cells separation according to the embodiments of the present invention, the concentration of the paramagnetic compound in the paramagnetic fluid and/or the magnet spacing in the magnetic levitation system may be adjusted to optimize purity and/or yield of the separation product (particle of interest).

In the embodiments of the cell separation methods that utilize multiple levitation-height altering and/or magnetic agents with different levitation-altering properties that are capable of forming complexes with different target cell types in a population of cells containing multiple cell types, more than two portions of the suspension may be formed after exposure to the magnetic field. For example, if a method utilizes two different levitation-height altering agents capable of binding to two different target cell types in a population of cells containing multiple cell types, two different types of complexes may be formed in a suspension upon exposure to the magnetic field: a first complex of first levitation-height altering agent and individual cells of a first target type, and a second complex of second levitation-height altering agent and individual cells of a second target type. In such a situation, the first levitation-height altering agent and the second levitation-height altering agent may be selected such that the levitation height of the first complex is different from the levitation height of the second complex, and is also different from the levitation height of the other types of cells in the population. In one example, at least three different portions of the suspension will then form in a processing channel of a flowcell cartridge upon its exposure to the magnetic field: a portion enriched by the first complex, a portion enriched by the second complex, and a portion (or portions) depleted of the first complex and the second complex. In this situation, the first and the second portions (which can be referred to as “fractions”) may require same or different length periods of exposure to the magnetic field to form. In one more example, upon exposure to the magnetic field, a first complex may “drop out” of the suspension to the bottom of the processing channel of the flowcell cartridge, and two different portions of the suspension will then form in the processing channel - a portion enriched by the second complex, and a portion depleted of the first complex and the second complex.

Cell separation methods according to the embodiments of the present invention may further include withdrawing different portions of the suspensions from the processing channel of the flowcell cartridge or from the flowcell cartridge altogether. Withdrawing of different portions or fractions can be performed through one or more outlet channels of the flowcell cartridge, and may involve flowing the suspension along the length of the processing channel.

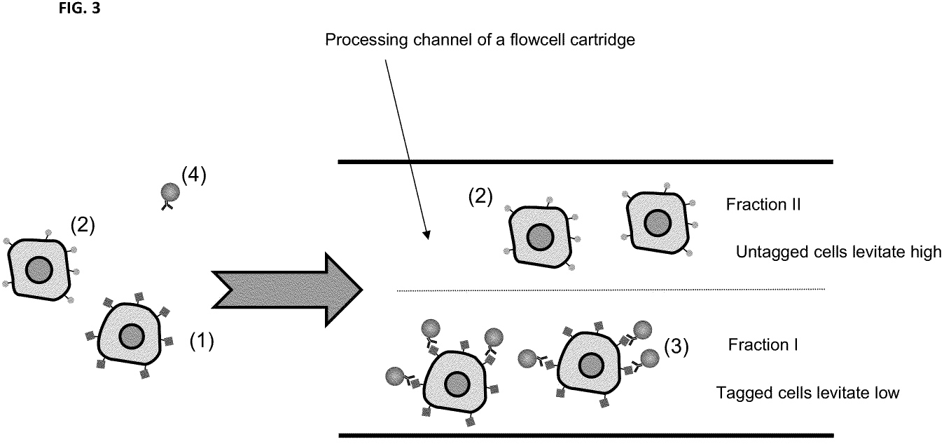

An exemplary embodiment of a cell separation method is schematically illustrated in FIG. 3. An embodiment illustrated in FIG. 3 is an example of a method of cell separation that uses magnetic microparticles with levitation-altering properties. The magnetic microparticles (which may be paramagnetic or superparamagnetic) are included in a levitation-height altering agent (4) capable of forming complexes with a first cell type (1), with the complexes (3) having lower levitation height than cells of the first type (1) that are not complexed. Levitation-height altering agent allows for separation of a fraction of a sample enriched in the complexes (3), or depleted of the complexes, thus allowing for separation of cells of a first type from a population of cells comprising multiple cell types. An exemplary population of cells contains at least two different cell types, a first type (1) and a second type (2), which have different surface markers (such as proteins, carbohydrates or other biological molecules). When an exemplary method is performed, the exemplary population of cells is contacted with the exemplary levitation-height altering agent (4) that comprises paramagnetic or superparamagnetic microparticles conjugated to antibodies capable of specifically binding to a surface marker of the first type (1) of the two cell types. The microparticles bind to the surface marker of the first cell type (1), forming complexes with the cells of the first type (1). When subjected to magnetic levitation, the complexes (3) levitate lower in the processing channel of the flowcell cartridge than the cells of the second type (2), which did not form the complexes with the microparticles. The fraction enriched in the complexes (fraction I) or the fraction depleted of the complexes (fraction II) is withdrawn from the flowcell cartridge, resulting in separation of cells of the first type and the second type.

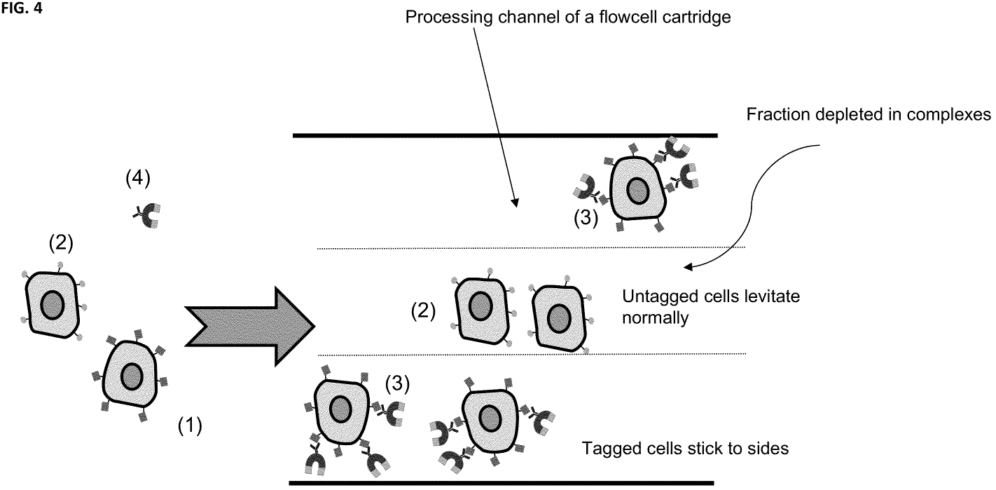

One more exemplary embodiment of a cell separation method schematically illustrated in FIG. 4. The embodiment illustrated in FIG. 4 is an example of a method of cell separation that uses magnetic microparticles that are immobilized to the sides of the processing channel of a flowcell cartridge when subjected to magnetic levitation. The cells that are not complexed with the magnetic microparticles levitate in the processing channel. An embodiment illustrated in FIG. 4 uses magnetic microparticles to deplete of a population of cells containing at least two cell types, a first type (1) and a second type (2), of the first type. The magnetic microparticles (which may be ferromagnetic, ferrimagnetic, paramagnetic or superparamagnetic) are included in a magnetic agent (4) capable of forming complexes with a first cell type (1), with the complexes (3) pulled by the magnetic force to the sides of the processing channel of the flowcell cartridge during magnetic levitation. Magnetic agent allows for separation of a fraction of a sample depleted of the complexes, thus allowing for separation of cells of the second type from a population of cells comprising multiple cell types. An exemplary population of cells contains at least two different cell types, a first type (1) and a second type (2), which have different surface markers (such as proteins, carbohydrates or other biological molecules). When an exemplary method is performed, the exemplary population of cells is contacted with the exemplary magnetic agent (4) that comprises magnetic microparticles conjugated to antibodies capable of specifically binding to a surface marker of the first type (1) of the two cell types. The microparticles bind to the surface marker of the first cell type (1), forming complexes with the cells of the first type (1). When exposed to magnetic field during magnetic levitation, the complexes (3) migrate to the sides of the processing channel of the flowcell cartridge. The fraction depleted of the complexes is withdrawn from the flowcell cartridge, resulting in separation of cells of the first type and the second type. In some embodiments, after the fraction depleted of the complexes of the first type is withdrawn from the flowcell cartridge, the exposure of the processing channel to the magnetic field may be stopped, and the complexes of the first type that migrated to one or more sides of the processing channel are then released into the processing channel, forming a suspension enriched with the complexes of the first type. The suspension enriched with the complexes of the first type may be then withdrawn from the flow cell cartridge, resulting in isolation of the cells of the first type (which form the part of the first complexes).

A magnetic agent according to the embodiments of the present invention may, but need not, include paramagnetic or superparamagnetic microparticles. Ferromagnetic or ferrimagnetic microparticles are also suitable for inclusion into magnetic agents according to the embodiments of the present invention and for use in the separation methods employing such magnetic agents. It is understood that levitation-altering agents comprising microparticles with superparamagnetic or paramagnetic properties may act, under suitable conditions, as magnetic agents and be used in the methods illustrated by the exemplary description above. For example, and as discussed elsewhere in the present disclosure, with the increase in the number of superparamagnetic or paramagnetic microparticles linked to each cell in complexes of cells with a levitation-height altering agent, the complexes may be immobilized against the bottom of the processing channel (“drop out” of the suspension) during a magnetic levitation process).

### Systems and Kits for Particle Separation

Described in the present disclosure and included among the embodiments of the present invention are kits and systems useful for separation of particles, such as cells, by magnetic levitation. An exemplary kit comprises one or more types of levitation-height altering agents and/or magnetic agents (as described in detail elsewhere in the present disclosure), or separate components of the one or more types of the levitation-height altering agents and/or the magnetic agents. Each levitation-height altering agent or magnetic agent is capable of forming complexes with individual particles, such as cells. Each levitation-height altering agent or magnetic agent comprises a magnetic microparticle and a linking agent that preferentially binds to a target cell type. For a levitation-height altering agent, the magnetic microparticle can be paramagnetic or superparamagnetic. For a magnetic agent, the magnetic microparticle can be ferromagnetic, ferrimagnetic, paramagnetic or superparamagnetic. In embodiments of kits that comprise one or more types of magnetic microparticle and one or more types of the linking agent as separate components, the one or more types of the magnetic microparticle may be surface-modified with moieties capable of mediating non-covalent interactions with the one or more types of the linking agent. The kit may also include one or more linking agents. Embodiments of a kit can may include a paramagnetic fluid medium. Embodiments of a kit may also include one or more of the other components, such as, but not limited to, antibodies, conjugating agents, buffers (including, but not limited to, buffers formulated to decrease non-specific binding of particles to non-target cells), flow cell cartridges, or materials designed to optimize depletion or recovery of target particles (such as cells) from a mixed particle population.

An exemplary system for separation of particles is a system of cell separation, which includes one or more types of levitation-height altering agents and/or magnetic agents (as described in detail elsewhere in the present disclosure). Each levitation-height altering agent and/or magnetic agent is capable of forming complexes with individual cells. Each levitation-height altering agent or magnetic agent comprises a magnetic microparticle and a linking agent that preferentially binds to a target cell type. For a levitation-height altering agent, the magnetic microparticle can be paramagnetic or superparamagnetic. For a magnetic agent, the magnetic microparticle can be ferromagnetic, ferrimagnetic, paramagnetic or superparamagnetic. In addition to one or more types of levitation-height altering agents and/or magnetic agents, an exemplary system includes a paramagnetic fluid medium. An exemplary system further includes a flowcell cartridge described elsewhere in the present disclosure, a station comprising a holding block for the flowcell cartridge, and one or more magnets positioned to expose the processing channel of the flowcell cartridge located in the holding block to a magnetic field.

### EXAMPLES

The following examples are offered to illustrate, but not to limit the claimed invention.

### Example 1: Separation of Jurkat Cells Using Magnetic Beads

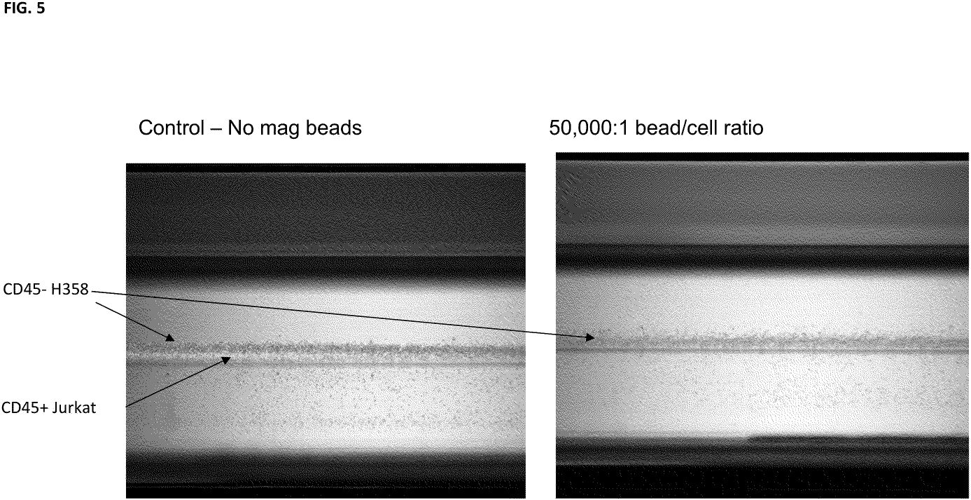

Separation of Jurkat cells (“Jurkat”) using microbeads was accomplished and is illustrated in FIG. 5. Streptavidin-coated high iron superparamagnetic nanobeads (300 nm diameter) were obtained from Spherotech (Lake Forest, Illinois, USA). CD45 anti-human antibody was bound to the microbeads. 15 µL of human anti-CD 45 antibody (clone 2D1, Biolegend, San Diego, California) was mixed with 9 µL of 5 µM free biotin. The resulting mixture was then mixed with 35 µL of the superparamagnetic nanobeads and incubated at room temperature for 30 minutes. At the end of the incubation period, the reaction was quenched with by adding an excess of 1 µM biotin and subsequently incubating for 5 minutes to fully bind all free biotin binding sites on the nanobeads. The nanobeads were then washed 3 times with 1x PBS/0.5% BSA to remove all unbound antibody and biotin, resulting in nanobeads with bound anti-human CD45 biotinylated antibody. Since the nanobeads had incubated with a solution containing 90% free biotin and 10% biotinylated antibody approximately 90% of the potential biotin binding sites on the nanobeads were bound to biotin, and 10% were bound to the biotinylated antibody. Jurkat cells of a human T-cell line (ATCC TIB-152, Jurkat clone E6.1) were stained with Calcein AM (Thermo Fisher Scientific Inc.) at 10 µM final concentration to provide green fluorescence. H358 cells (NCI-H358 obtained from Berkeley Cell Culture Facility, Berkeley, California) were stained with CellTracker Red CMPTX (Thermo Fisher Scientific Inc.) at 10 µM final concentration to provide red fluorescence. Both types of stained cells were washed 3 times to remove excess fluorescence stain. Stained Jurkat and H358 cells were mixed to ^{~}50% H358 cells/^{~}50% Jurkat cells (“H358/Jurkat mixture”). The nanobeads with bound anti-human CD45 biotinylated antibody were added to the H358/Jurkat mixture at 10⁶ total cell input, meaning that there were about 1 million total cells, with about 500,000 Jurkat cells and about 500,000 H358 cells. The nanobeads with bound anti-human CD45 biotinylated antibody were added to this cell mixture, and the resulting mixture was incubated for 15 minutes at 25° C. 50,000:1 bead-to-cell ratio input, meaning that about 25x10⁹ nanobeads with bound anti-human CD45 biotinylated antibody were added to H358/Jurkat mixture. Paramagnetic fluid medium containing 1 M gadobutrol was then added to the cell/nanobeads mixture to a final concentration of 75 mM . The resulting levitation suspension was loaded into a flowcell cartridge of LeviCell™ magnetic levitation platform (Levitas Bio, Menlo Park, California, USA). The levitation suspension was exposed to magnetic field (“equilibrated”) for 20 minutes inside the LeviCell instrument, and then flowed through the flowcell. A control experiment was conducted without the addition of magnetic beads.

FIG. 5 shows photographic images (reproduced in greyscale) of the cell mixture at the end of the equilibration phase in the flowcell for the control experiment (left image) and the mixture with the magnetic beads (right image). As shown in the image obtained from the controlled experiment, both CD45+ Jurkat cells (labeled with green fluorescent Calcein AM dye) and CD45- H358 cells (labeled with red fluorescent CellTracker Red dye) were levitating at about the same position in the flow cell. In the 50,000:1 bead-to-cell ratio image, the CD45+ Jurkat cells (green fluorescence) are no longer levitating because the magnetic particles bound to the cells have pulled the cells out of solution (depleted the cells) and the CD45- H358 cells are still levitating at the same position as they did in the control reaction. The results illustrated in FIG. 5 showed 99.87% depletion of Jurkat CD45+ cells, via Nexcelom cell counter. To determine the level of depletion, output samples were collected from the flowcell cartridge, and their volumes were measured using a micropipette. A 20 µl sample was placed into a cell counter chip (Nexcellom Bioscience, Lawrence, Massachusetts) and placed in Nexcelom Cellometer instrument. Green-labelled Jurkat cells and red-labelled H358 cells were automatically counted, and the concentrations of each type of cell were calculated. The calculated concentrations were multiplied by the sample volume to arrive at the total number of cells in each output sample, as well as in each input sample set aside earlier. The depletion level was calculated as the inverse ratio of the total amount of Jurkat cells in the top output fraction to the input fraction:

$$D e p l e t i o n = \left(1 - \frac{l i v e C D {45}^{p o s} c e l l s i n t o p}{l i v e C D {45}^{p o s} c e l l s i n i n p u t}\right) .$$

No depletion was observed in the control experiment.

### Example 2: Comparison of the Different Complex Formation Conditions

Separation of Jurkat cells using magnetic microbeads was performed substantially as described in Example 1 with the following modifications. Two different complex formation conditions were tested. Under the first set of conditions of streptavidin-conjugated the superparamagnetic nanobeads were mixed with the cells already labeled with the biotinylated antibody. Under the second set of conditions, the superparamagnetic nanobeads were first conjugated to the antibody, and then mixed with the cells. In the first experimental scenario, a mixture of 15% CD45^{neg}/85% CD45^{pos} cells were labeled with a saturating concentration of biotin-conjugated anti-human CD45 antibody to bind all available CD45 on the surface of the cells. Excess antibody was washed away and the labeled cells were incubated with streptavidin-conjugated superparamagnetic nanobeads. In the second experimental scenario, the streptavidin-conjugated superparamagnetic nanobeads were mixed with biotin-conjugated antibody, and the remaining free streptavidin was blocked by free biotin. The antibody-linked superparamagnetic nanobeads were then incubated with the cell mixture to bind to CD45. Due to the lower affinity of the antibody-CD45 interaction (K_{D} of approximately 10⁻⁶ to 10⁻⁹ mol/L) compared to streptavidin-biotin interaction (K_{D} of approximately 10⁻¹⁵ mol/L), nearly 10x superparamagnetic nanobeads were required to achieve similar CD45^{pos} cell depletion levels. Due to the number of beads required, it was not feasible to label all available biotin binding sites with antibody due to the cost involved, which is why free biotin was used to block unbound binding sites on the beads.

A certain amount of a linking agent bound per microparticle is needed to reach the saturating “solution phase” concentration of the linking agent to maximize binding to the cells being tagged. Completely covering the surface of the microparticles with a linking agent would be ideal. However, there can be a large number of potential ligand binding sites on microparticle surface, which may require an impractically high amount of the linking agent to effect complete surface coverage. In place of complete surface coverage, in some cases there is a satisfactory minimum required amount of the linking agent bound to the microparticle surface to provide sufficient “solution phase” concentration of the linking agent to bind sufficiently high proportion of the cells being tagged. Since leaving free binding sites on the microparticles may increase non-specific binding of the microparticles to cells, adding a “filler” or “blocking” agent to cover these binding sites may be advantageous.

### Example 3: The Effect of Microparticle Size on Cell Separation

It was theoretically predicted and experimentally confirmed that the most effective binding of the levitation-height altering agent or magnetic agent to cells was performed when using the microparticles of smallest size possible, such that the product of the number of the linking agent molecules per microparticle and the number of the microparticles per unit solution volume is as close as possible to the saturating concentration of that particular linking agent in solution. For example, two different types of microparticles made from the same material and having the same density can have different sizes, the first microparticle 5 µm in diameter, and the second microparticle 1 µm in diameter. The 5 µm microparticle has 125x the particle volume and 25x the surface area compared to the 1 µm microparticle. Assuming that a fixed surface area required for every antibody binding event, the 5 µm microparticle can bind 25x linking agent molecules than the 1 µm microparticle. However, in solution, at the same weight-per-volume concentration of microparticles, the 1 µm microparticle will be 125x more concentrated. Thus, the effective solution concentration of the microparticle-bound linking agent is 5x higher for the 1 µm microparticles than for the 5 µm microparticles, with the same weight-per-volume concentration. Thus, it is easier to get to saturating concentration of microparticle-bound linking agent in solution by using smaller microparticles and using a higher number of microparticles per unit volume. It was experimentally determined that, to achieve effective cell separation on LeviCell® magnetic levitation platform, the size of the microparticles has to be small enough to reach saturating concentration in solution, and large enough to contain enough magnetic material (such as Fe₃O₄) to be move the complexes formed with the cells downwards towards the fixed magnet and/or and to effectively alter the density of the complex of the cell to effect its levitation height. The effect of the microparticle size on the density of the complex of the cell with the density-modifying agent is illustrated in FIG. 8, which is discussed elsewhere in the present disclosure.

### Example 4: Testing of the Effects of Antibody Surface Coverage on Cell Separation

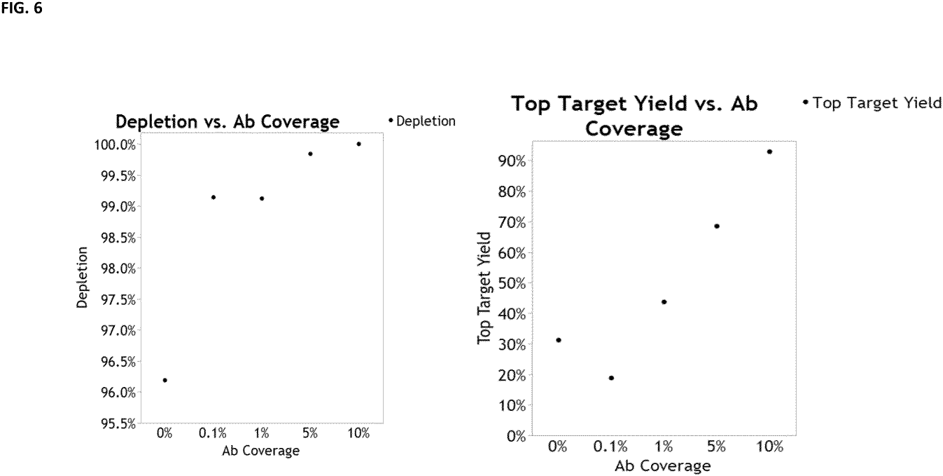

Separation of Jurkat cells using magnetic microbeads was performed substantially as described in Example 1 with the following modifications. Streptavidin-coated nanobeads were incubated with varying amounts of biotinylated anti-CD45 antibody to cover different percentages of available streptavidin binding sites (the binding site data was provided by the manufacturer). The remaining binding sites were blocked with biotinylated BSA and/or free biotin. The resulting nanobeads with bound anti-human CD45 biotinylated antibody were incubated with a cell mixture of 15% CD45^{neg} cells/85% CD45^{pos} cells, and magnetic levitation separation process was performed. The effectiveness of the separation process was evaluated by measuring the depletion of CD45^{pos} cells and the yield of CD45^{neg} cells from the sample. The results, which are illustrated in FIG. 6, showed that 10% antibody coverage of the beads was sufficient to provide >99.9% depletion of CD45^{pos} cells while maintaining a >50% yield of CD45^{neg} cells. Reducing the antibody coverage directly reduced the depletion of CD45^{pos} cells and the yield of CD45^{neg} cells, possibly due to an increase in non-specific binding.

### Example 5: Testing of Different Microparticle Materials

A variety of microparticle materials have been tested in cell separation processes according to the exemplary embodiments described in the present disclosure. Some of the microparticles tests showed more non-specific binding than the others. Non-specific binding was primarily observed with the microparticles with an iron/polymer, silica or gold coating. Microbeads sourced from Creative Diagnostics (New York, New York) exhibited low non-specific binding, as expected since the manufacturer explicitly stated that the microbeads were blocked to reduce non-specific binding. 100 nm superparamagnetic high iron beads obtained from Creative Diagnostics showed >99% depletion when the cells were labeled with antibody before mixing with beads at 1000 bead-to-cell ratio. Conjugating the microparticles with antibody prior to mixing with the cells resulted in depletion of 87% at 10,000 bead-to-cell ratio. Conjugating the microparticles with antibody prior to mixing with the cells reduced the minor non-specific binding observed.

Initial testing of magnetic beads obtained from Spherotech (Lake Forest, Illinois, USA). involved 5 cm and 0.5 µm paramagnetic microparticles having a polystyrene/iron oxide shell around a polystyrene core and is marketed as having “regular iron content.” These paramagnetic microparticles showed significant non-specific binding to all cells, regardless of the size. Smaller Spherotech microparticles (0.5 µm and 0.3 µm) marketed as “high iron” were also tested. These high-iron microparticles have an iron oxide core and a polystyrene shell. The 0.5 µm microparticles showed little non-specific binding, but did not achieve >90% depletion. The difference of specificity between the high and low iron content 0.5 µm beads showed the importance of material selection (such as microparticle coating and iron content) for performance of the beads for depletion. The 0.3 µm high iron content beads achieved >95% depletion.

Ferrofluid obtained from Bio-Techne (Minneapolis, Minnesota) contains 100-300 nm superparamagnetic microparticles with a polymer coating. U.S. Pat. No. 7,169,618 suggests multiple coating possibilities for these microparticles, including silanization and carboxydextran or aminodextran coating. Direct conjugation of antibody or streptavidin to the microparticle surface is also envisioned. Streptavidin conjugation of biotinylated antibodies to the microparticles was performed. Since the biotin binding capacity of the microparticles was unknown, a titration of streptavidin conjugated beads with a set amount of biotinylated antibody was performed first. A depletion of >99% of CD45^{pos} cells was achieved with 20 µl of ferrofluid labeled with 25 µl of biotinylated antibody. When 400 µl of ferrofluid was labeled with 25 µl of biotinylated antibody, a reduction in depletion to about 85% was observed, suggesting that not enough antibody was present to achieve sufficient antibody levels on the microparticle surface. 50 µl ferrofluid samples were used for titration of the antibody concentration. Depletion of nearly 100% of CD45^{pos} cells was observed for all tested antibody volumes ranging from 25 µl to 6.25 µl. The ferrofluid showed better depletion performance when the cells were incubated with the antibody first, achieving >99.5% depletion. Minimal non-specific binding was observed when labeling with an isotype control or no antibody. These beads were not used for further testing as it was unavailable for production. However the patent recently expired, allowing for potential production of these beads.

BD IMag® microparticles were sourced from BD Biosciences (San Jose, California). These microparticles have a superparamagnetic iron oxide core with a polymer shell. Initial testing used streptavidin conjugated microparticles with biotinylated antibody to specifically target CD45^{pos} cells. Since the biotin-binding capacity of the beads was unknown, the cells were first stained with antibody, washed, and then mixed with streptavidin conjugated microbeads. This resulted in nearly 100% depletion and good yield of the desired cell population.

Dextran CLIO magnetic nanoparticles were obtained from Luna Nanotech (Markham, Ontario, Canada). These nanoparticles are composed of cross-linked dextran with one to three 7-14 nm iron oxide spheres inside each nanoparticles. Using nanobeads conjugated to anti-CD45 antibody, average depletion of about 92% was achieved with some significant non-specific binding observed.

Gold coated nanoparticles with iron oxide cores were obtained from Nanopartz® (Loveland, Colorado). CD45 anti-human antibody was bound 100 nm streptavidin conjugated nanoparticles and tested in a depletion experiment. About 98% depletion was achieved, however non-specific binding was observed to all cells when using beads without antibody, quenched with free biotin. In subsequent experiments, some non-specific binding to the CD45^{Neg} cells by the anti-CD45 coated beads at bead-to-cell ratios of >50,000:1 was observed. Further testing with alternative blocking agents would be needed to make feasible for depletion.

It is understood that the examples and embodiments described in the present disclosure are for illustrative purposes only and that various modifications or changes in light thereof will be suggested to persons skilled in the art and are to be included within the spirit and purview of this application and scope of the appended claims. All publications, patents, and patent applications cited in the present disclosure are hereby incorporated by reference in their entirety for all purposes.

What is claimed is:

1. A method of cell separation, comprising:
a) combining a magnetic agent and population of cells comprising multiple cell types,
wherein the magnetic agent comprises a magnetic microparticle and a linking agent that preferentially binds to cells of a target type of the multiple cell types, thereby forming a magnetic complex, said magnetic complex comprising the magnetic agent bound to an individual cell of the target type;
b) forming a suspension in a paramagnetic fluid medium, the suspension comprising a plurality of the magnetic complexes and a plurality of the cells of the multiple cell types;
c) introducing the suspension into a processing channel of a flowcell cartridge; and,
d) exposing the processing channel to a magnetic field for a period of time sufficient for at least some of the plurality of the magnetic complexes to migrate to and be immobilized against one or more sides of the processing channel, thereby forming a suspension depleted of the magnetic complex.

2. The method of claim 1, wherein the linking agent comprises an antibody or a domain of the antibody capable of specifically binding to a moiety on a surface of the cell of the target type.

3. The method of claim 2, wherein the binding of the magnetic agent comprises:
binding of the antibody or a domain of the antibody to the moiety on a surface of the cell of the target type; and,
binding of the antibody or the domain of the antibody to the magnetic microparticle.

4. The method of claim 1, wherein the linking agent binds to the magnetic microparticle by one or more covalent or non-covalent interactions.

5. The method of claim 1, wherein the cell of the target type is selected from the group consisting of macrophages, alveolar type II (ATII) cells, stem cells, adipocytes, cardiomyocytes, embryonic cells, tumor cells, lymphocytes, red blood cells (erythrocytes), epithelial cells, ova (egg cells), sperm cells, T cells, B cells, myeloid cells, immune cells, hepatocytes, endothelial cells, stromal cells, and bacterial cells.

6. The method of claim 2, wherein the moiety on a surface of the cell of the target type is selected from the group consisting of CD45, CD3, CD4, CD8, CD19, CD40, CD56, CD11b, CD14, CD15, EpCAM, ICAM, CD235, HER-2, HER-3, CD66e, Integrins, E- P- L-Selectins, EGFR, EGFRVIII, PDGFR β, c-MET, MUC-1, OX-40, CD28, CD133, CD30 TNFRSF8, CTLA4, CD71, CD16α VCAM-1, Nucleolin, and Myelin Basic Protein.

7. The method of any one of claim 1, further comprising:
e) withdrawing at least part of the suspension depleted of the magnetic complex from the flowcell cartridge.

8. The method of claim 7, further comprising, after performing step (e):
f) stopping the exposing of the processing channel to the magnetic field, thereby releasing the magnetic complexes immobilized against the one or more sides of the processing channel, thereby forming a suspension enriched with the magnetic complex; and,
g) withdrawing at least part of the suspension with the magnetic complex from the flowcell cartridge.

9. The method of method of claim 8, wherein step (e) or (g) is performed through an outlet channel of the flowcell cartridge.

10. The method of claim 1, wherein the magnetic microparticle is a paramagnetic, superparamagnetic, ferromagnetic, or ferrimagnetic microparticle.

11. The method of claim 10, wherein the magnetic microparticle comprises a metal, a metal salt, or a metal oxide.

12. A method of cell separation, comprising:
a) binding a first levitation-height altering agent to a cell of a first type in a population of cells comprising multiple cell types, wherein the first levitation-height altering agent comprises a first paramagnetic or superparamagnetic microparticle and a first linking agent that preferentially binds to cells of the first type, thereby forming a first complex, said first complex comprising the first levitation-height altering agent bound to an individual cell of the first type;
b) forming a suspension in a paramagnetic fluid medium, the suspension comprising a plurality of the first complexes and a plurality of the cells of the multiple cell types;
c) introducing the suspension into a processing channel of a flowcell cartridge; and,
d) exposing the processing channel to a magnetic field for a first period of time sufficient for at least some of the first complexes to separate in the processing channel from the cells of
the multiple cell types not bound by the first levitation-height altering agent, thereby forming a first portion of the suspension, wherein the first portion is enriched with the first complex relative to the suspension, and a second portion of the suspension, the second portion depleted of the first complex relative to the suspension.

13. The method of claim 12, wherein the first complex levitates lower in the processing channel of the flowcell cartridge than the multiple cell types not bound by the first levitation-height altering agent.

14. The method of claim 12, wherein the first linking agent comprises a first antibody or a domain of the first antibody capable of specifically binding to a first moiety on a surface of the cell of the first type.

15. The method of claim 14, wherein the binding of the first levitation-height altering agent comprises:
binding of the first antibody or a domain of the first antibody capable of specifically binding to the first moiety on a surface of the cell of the first type, to the first moiety on a surface of the cell of the first type; and,
binding of the first antibody or the domain of the first antibody to the first paramagnetic or superparamagnetic microparticle.

16. The method of claim 15, wherein the cell of the first type is selected from the group consisting of macrophage, alveolar type II (ATII) cell, stem cell, adipocyte, cardiomyocyte, embryonic cell, tumor cell, lymphocyte, erythrocytes, epithelial cell, egg cell, sperm cell, T cell, B cell, myeloid cell, immune cell, hepatocyte, endothelial cell, stromal cell, and bacterial cell.

17. The method of claim 15, wherein the first moiety is selected from the group consisting of CD45, CD3, CD4, CD8, CD19, CD40, CD56, CD11b, CD14, CD15, EpCAM, ICAM, CD235, HER-2, HER-3, CD66e, Integrins, E- P- L-Selectins, EGFR, EGFRVIII, PDGFR β, c-MET, MUC-1, OX-40, CD28, CD133, CD30 TNFRSF8, CTLA4, CD71, CD16α VCAM-1, Nucleolin, and Myelin Basic Protein.

18. A magnetic levitation kit comprising a paramagnetic fluid medium and one or more magnetic agents, or separate components of the one or more of the magnetic agents, capable of forming complexes with individual cells,
wherein each magnetic agent comprises a magnetic microparticle, and a linking agent that preferentially binds to a target cell type.

19. The kit of claim 18, wherein the magnetic microparticle is a paramagnetic, superparamagnetic, ferromagnetic or ferromagnetic microparticle.

20. The kit of claim 19, wherein the linking agent preferentially binds to a cell surface marker selected from the group consisting of CD45, CD3, CD4, CD8, CD19, CD40, CD56, CD11b, CD14, CD15, EpCAM, ICAM, CD235, HER-2, HER-3, CD66e, Integrins, E- P- L-Selectins, EGFR, EGFRVIII, PDGFR β, c-MET, MUC-1, OX-40, CD28, CD133, CD30 TNFRSF8, CTLA4, CD71, CD16α VCAM-1, Nucleolin, and Myelin Basic Protein.
